(19) United States
(12) Patent Application Publication
(10) Pub. No.: US 2014/0329288 A1
(43) Pub. Date: Nov. 6, 2014

(54) IN VIVO CONVERSION OF LIGHT ENERGY INTO HYDROGEN GAS

(71) Applicant: Robert A. Ludwig, Santa Cruz, CA (US)

(72) Inventor: Robert A. Ludwig, Santa Cruz, CA (US)

(73) Assignee: The Regents of University of California, Oakland, CA (US)

(21) Appl. No.: 14/240,342

(22) PCT Filed: Aug. 31, 2012
(86) PCT No.: PCT/US2012/053539

§ 371 Date: Jun. 25, 2014

Related U.S. Application Data
(60) Provisional application No. 61/529,852, filed on Aug. 31, 2011.

(51) Int. Cl. C12P 3/00

(52) U.S. Cl. CPC C12P 3/00
USPC 435/168; 435/252.3; 435/320.1

(57) ABSTRACT

The present disclosure provides isolated phototrophic cells for producing hydrogen and methods for use thereof. The methods involve inducing endo-hydrogenase expression in the phototrophic cells through the use of a regulatable promoter and harvesting light energy for hydrogen production. In preferred embodiments the hydrogenase is a member of the Hyq family of endo-hydrogenases and the isolated phototrophic cell is a Rhodopseudomonas palustris cell.

### CROSS REFERENCE TO RELATED APPLICATIONS

This application is a U.S. National Phase of PCT/US2012/053539, filed Aug. 31, 2012, which claims the benefit under 35 USC 119(e) of U.S. Provisional Application No. 61/529,852, filed Aug. 31, 2011, each of which is incorporated herein by reference in its entirety for all purposes.

### FIELD

The present disclosure generally relates to methods and compositions for hydrogen production. In particular, the present disclosure relates to hydrogen production through use of recombinant phototrophic bacteria.

### BACKGROUND

In view of peaking oil production and accelerating global warming the need to develop clean, sustainable, and economically viable energy supplies is rapidly gaining in urgency. Hydrogen is an attractive alternative fuel source because hydrogen combustion produces water instead of greenhouse gases as an end product. However, a viable hydrogen economy relies on clean, sustainable, and economic ways of generating hydrogen. Current hydrogen production depends heavily, if not exclusively, on the use of non-renewable resources, such as steam reformation of natural gas, coal gasification and nuclear-power-driven electrolysis of water. While these approaches may facilitate the transition towards a hydrogen economy, the hydrogen currently produced is more expensive and contains less energy than the non-renewable energy source from which it is made. Therefore, there is a clear need for new and greener means of producing hydrogen.

One attractive solution to the problem of green hydrogen production is the use of photosynthetic microorganisms as hydrogen factories. Photosynthesis is in essence the photoelectrolysis of water, an energy requiring process for the conversion of water (H₂O) into hydrogen (H₂) and oxygen (O₂). However, hydrogen production by wild type microorganisms is energetically wasteful and evolutionarily disfavored. Hydrogen is a volatile gas, which cannot be stored within living cells or their organelles. Therefore, any hydrogen produced by microorganisms dissipates, resulting in a loss of the harvested photoenergy.

Consequently, few microorganisms even possess the capability of hydrogen production. The few exceptional microorganisms that produce hydrogen do so in an inefficient manner. For instance, hydrogen-producing diazotrophic bacteria such as Azorhizobium caulinodans are organotrophes that obtain the energy required for hydrogen production through the oxidative metabolism of organic matter, and in the process consume oxygen while generating the greenhouse gas carbon dioxide (CO₂). Thus with respect to the energy and green house gas balance, hydrogen production by organotrophs resembles the traditional approaches of burning fossil fuels.

On the other hand, photosynthetic hydrogen producers that utilize light energy to drive the photoelectrolysis of water immediately utilize the captured light energy for the reduction of oxygen, and only indirectly leverage solar power for hydrogen production. This indirect coupling of solar power and hydrogen formation is energetically unfavorable. As a result wild type phototrophes at best utilize 15 to 20% of their cellular photosynthetic capacity for hydrogen production (e.g., Mellis and Happe, Plant Physiol. 127:740-48, 2001).

Therefore a clear need exists for optimized cells and processes for effectively producing hydrogen from light.

### BRIEF SUMMARY

The present disclosure generally relates to methods and compositions for hydrogen production. In particular, the present disclosure relates to hydrogen production through use of recombinant phototrophic bacteria.

Specifically, the present disclosure provides recombinant, phototrophic bacterium comprising: a) a nucleic acid encoding structural proteins of an endo-hydrogenase operon; b) a heterologous nucleic acid encoding a transcriptional repressor/activator protein responsive to an inducer; and c) a heterologous nucleic acid comprising bidirectional promoter regions; wherein the heterologous nucleic acid comprising bidirectional promoter regions is located in operable combination with the nucleic acid encoding the structural proteins on one side and the heterologous nucleic acid encoding the transcriptional repressor/activator protein on another side, and wherein the transcriptional repressor/activator induces expression of the structural proteins when the recombinant bacterium is cultured in the presence of the inducer. In some embodiments, the nucleic acid encoding the structural proteins of the endo-hydrogenase operon is endogenous to the bacterium. In some embodiments, the bacterium is an alpha-proteobacteria. In a subset of these embodiments, the alpha-proteobacteria is a Rhizobiales bacterium such as Rhodopseudomonas palustris, Rhodospirillum centenum or Azorhizobium caulinodans. In some embodiments, the alpha-proteobacteria is a Rhodopseudomonas palustris strain in which the endo-hydrogenase operon is a hyq operon that is repressed when the alpha-proteobacteria is shifted to phototrophic culture. In some embodiments, the nucleic acid encoding the structural proteins of the endo-hydrogenase operon is heterologous to the bacterium. In some embodiments, the structural proteins of the endo-hydrogenase operon are at least 70%, 75%, 80%, 85%, 90%, 95%, 96%, 97%, 98%, 99%, or 100% identical to Rhodopseudomonas palustris structural proteins of SEQ ID NOS:10-15, SEQ ID NOS:16-21, SEQ ID NOS:22-27, and SEQ ID NOS:28-33. In some embodiments, the structural proteins of the endo-hydrogenase operon are at least 70%, 75%, 80%, 85%, 90%, 95%, 96%, 97%, 98%, 99%, or 100% identical to Azorhizobium caulinodans structural proteins of SEQ ID NOS:3-8. In some embodiments, the bacterium is a cyanobacterium. In a subset of these embodiments, the cyanobacterium is selected from the group consisting of Synechococcus sp. and Synechocystis sp. In some embodiments, the nucleic acid encoding the structural proteins of the endo-hydrogenase operon is codon-optimized for expression in the cyanobacterium (e.g., employing the codon-usage table for Synechococcus elongatus). In some embodiments, the structural proteins of the endo-hydrogenase operon comprise HyqB, HyqC, HyqE, HyqF, HyqG and HyqI. In some embodiments, the heterologous nucleic acid encoding the transcriptional repressor/activator protein and the heterologous nucleic acid comprising the bidirectional promoter regions comprise Pseudomonas putida nahR coding region and Pseudomonas putida nahR/nahG promoter regions, and the inducer comprises salicyclic acid.

Also provided by the present disclosure are methods for producing hydrogen photosynthetically, the methods comprising: a) growing the recombinant bacterium of the preceeding paragraph phototrophically in media in the absence of the inducer to produce a bacterial culture; b) supplementing the media of the bacterial culture with the inducer; and c) exposing the bacterial culture with light in the presence of the inducer so as to express the structural proteins of the endo-hydrogenase operon, thereby producing H₂ gas photosynthetically from the bacterial culture. In some embodiments, the bacterial culture produced in step a) is a late-exponential phase bacterial culture. In some embodiments, the steps a) through c) take place under microaerobic conditions (e.g., micromolar O₂). In some embodiments, the microaerobic conditions comprise sparging the bacterial culture with nitrogen. In some embodiments, the methods further comprise step d) extracting the H₂ gas from gas streams from the nitrogen-sparged bacterial cell culture. In some embodiments, the step b) further comprises supplementing the media with one or more of the group consisting of thiosulfate, sulfite, dimethyl sulfide, and trimethylamine. In some embodiments, step c) further comprises subjecting the bacterial culture to an electric current. In some embodiments, step c) further comprises preparing an emulsion comprising magnetite nanoparticles, latex and bacteria from the bacterial culture; applying the emulsion to graphite electrode surface; and subjecting bacteria in the emulsion to an electric current conducted through the electrode surface. Additionally or alternatively, other biological electron-donors themselves incapable of producing molecular hydrogen in living cells from hydrogen-ions donated by water absent net energy input are provided (e.g., ultraviolet, visible, or infrared light). In some embodiments, the light comprises sun light. In some embodiments, the light comprises filtered light of a wavelength range optimized for H₂ production. In some embodiments, the bacterial cell culture is a liquid culture. In other embodiments, the bacterial cell culture is a solid culture.

In addition, the present disclosure provides expression cassettes comprising: a) a nucleic acid encoding structural proteins of an endo-hydrogenase operon; b) a heterologous nucleic acid encoding a transcriptional repressor/activator protein responsive to an inducer; and c) a heterologous nucleic acid comprising bidirectional promoter regions; wherein the heterologous nucleic acid comprising bidirectional promoter regions is located in operable combination with the nucleic acid encoding the structural proteins on one side and the heterologous nucleic acid encoding the transcriptional repressor/activator protein on another side, and wherein the transcriptional repressor/activator induces expression of the structural proteins in the presence of the inducer. The present disclosure further provides vectors comprising the expression cassette, a bacterial origin of replication, and a coding region of a selectable marker in operable combination with a regulatory sequence. In some embodiments, the structural proteins of the endo-hydrogenase operon comprise HyqB, HyqC, HyqE, HyqF, HyqG and HyqI. In some embodiments, the endo-hydrogenase operon is an alpha-proteobacterial endo-hydrogenase operon. In some embodiments, the alpha-proteobacteria is Rhodopseudomonas palustris or Azorhizobium caulinodans. In some embodiments, the structural proteins of the endo-hydrogenase operon are at least 70%, 75%, 80%, 85%, 90%, 95%, 96%, 97%, 98%, 99%, or 100% identical to Rhodopseudomonas palustris structural proteins of a strain selected from the group consisting of HaA2, BisA53, BisB5 and BisB18 (e.g., SEQ ID NOS: 10-15, 16-21, 22-27 or 28-33). In some embodiments, the structural proteins of the endo-hydrogenase operon are at least 70%, 75%, 80%, 85%, 90%, 95%, 96%, 97%, 98%, 99%, or 100% identical to Azorhizobium caulinodans structural proteins of SEQ ID NOS:3-8. In some embodiments, the nucleic acid encoding the structural proteins of the endo-hydrogenase operon is codon-optimized for expression in cyanobacteria (e.g., employs the codon-usage table for Synechococcus elongatus). In some embodiments, the heterologous nucleic acid encoding the transcriptional repressor/activator protein and the heterologous nucleic acid comprising the bidirectional promoter regions comprise Pseudomonas putida nahR coding region and Pseudomonas putida nahR/nahG promoter regions, and the inducer comprises salicyclic acid. The present disclosure also provides kits for inducible expression of an endo-hydrogenase in a phototrophic bacterial host cell, comprising the vector described above, and a small molecule inducer. Moreover the present disclosure provides methods for producing a recombinant, phototrophic bacterium for producing hydrogen photosynthetically, the method comprising: introducing the vector described above into a phototrophic bacterial cell to produce a recombinant, phototrophic bacterium. In some embodiments, the phototrophic bacterium is an alpa-proteobacterium or a cyanobacterium.

### BRIEF DESCRIPTION OF THE DRAWINGS

FIG. 1 illustrates the structure-function model of L-type Hyq endo-hydrogenases by analogy and homology to respiratory complex I. Inferred membrane quinone (Q) binding at the interface of HyqC, HyqG, and HyqI requires quinone partial (14 Å) extraction from the respiratory membrane hydrophobic phase (Efremov et al., Nature 465:441-45, 2010). The location of HyqG catalytic sites remain to be determined in detail; in vivo activity is reversible.

FIG. 2A-F provides an alignment of the amino acid sequences of the Hyq structural proteins of four wild type isolates of R. palustris. HyqB, HyqC, HyqE, HyqF, HyqG and HyqI of R. palustris strain HaA2 are set forth as SEQ ID NOs:10-15, HyqB, HyqC, HyqE, HyqF, HyqG and HyqI of R. palustris strain BisA53 are set forth as SEQ ID NOs:16-21, HyqB, HyqC, HyqE, HyqF, HyqG and HyqI of R. palustris strain BisB5 are set forth as SEQ ID NOs:22-27, and HyqB, HyqC, HyqE, HyqF, HyqG and HyqI of R. palustris strain BisB18 are set forth as SEQ ID NOs:28-33.

FIG. 3A-F provides an alignment of the amino acid sequences of the Hyq structural proteins of A. caulinodans versus their Nuo (complex I) homologs: NuoL (SEQ ID NO:34), NuoH (SEQ ID NO:35), NuoJ (SEQ ID NO:36), NuoM (SEQ ID NO:37), NuoC (SEQ ID NO:38), NuoD (SEQ ID NO:39) and NuoB (SEQ ID NO:40).

### DETAILED DESCRIPTION

Photosynthesis, in essence, is the photoelectrolysis of water, an energy requiring process. Living photosynthetic creatures supply and sustain the necessary voltage to the electron transfer circuit of photosynthetic membranes by harvesting solar energy. Serving as continuously recharging “battery” for the biological photoelectrolytic circuit is the core light-harvesting complex (LHC); each LHC inputs photons and outputs accelerated electrons, conserving energy as a quantum coupled process. Indeed, the LHC in crystalline form is capable of unabated light-to-electron energy conversion at 4.2° K (approaching absolute zero) and so may be considered a solid-state physical device (Chachisvilis et al., Chemical Physics Letters, 224:345-354, 1994; and Shreve et al., Biochim Biophys Acta Bioenerg, 1058:280-288, 1991).

Chemically, the electrolytic cell produces from water, molecular oxygen (O₂) at the low chemical (high electrical) potential electrode. The high chemical (low electrical) potential electrode yields molecular hydrogen (H₂), in which electrochemical energy is largely conserved. All cyanobacteria and higher (algae and vascular plants) living photosynthetic creatures are capable of oxygenic photoelectron transport. Yet, none are hydrogenic. H₂, the most volatile of all molecules, cannot be contained in living photosynthetic creatures, cells, and cellular compartments (chloroplasts). Any H₂ so produced and dissipated as hydrogenic photoelectron transport would essentially represent complete loss of photoenergy transduction and conservation.

Instead, as a biological process, oxygenic photoelectron transport produces, traps, and stores in living cells energized hydrogen (anion) as NADPH (nicotinamide adenine dinucleotide phosphate), in which the “H” appended to the molecule's acronym signifies its chemical form representing stored hydrogen (i.e., electrochemical) energy. In purple bacteria, however, the photoelectron transport occurring as part of photosynthesis does not yield a strong reductant, such as NADPH, for CO₂ fixation. Rather, purple bacterial photosynthesis represents cyclic photophosphorylation (ATP resynthesis), a light energy-dependent process.

In diverse microaerophilic a-proteobacteria, a membrane-integral endo-hydrogenase enzyme activity recycles endogenous molecular hydrogen (H₂) produced during biological fixation of N₂, the predominant constituent of earth's modern atmosphere. This endo-hydrogenase is genetically encoded in a six- or seven-gene hyq operon whose six, structural proteins are all homologs of constituent proteins in NADH:quinone oxidoreductase, commonly known as respiratory complex I. In bacteria, the respiratory complex I is typically a 14-protein integral membrane complex (Ng et al., PLoS ONE 4:e4695, 2009; and Ciccolella et al., PLoS ONE 5:e12094, 2010). In aerobic and microaerophilic bacteria, most respiratory hydrogenases are classified as group I, the unidirectional, uptake hydrogenases, whose catalytic site faces the cell exterior (exo-hydrogenases). However, the Hyq hydrogenase is classified as a bidirectional group 4 enzyme, capable of both breaking and making H₂. While also membrane-integral in bacterial cells, each Hyq hydrogenase catalytic site faces the cell interior (endo-hydrogenases).

The present disclosure generally relates to methods and compositions for endo-hydrogenase-mediated hydrogen production. In particular, the present disclosure relates to hydrogen production through use of recombinant phototrophic cells.

### Vectors, Systems and Kits for the Inducible Expression of Hydrogenases

In one embodiment the present disclosure provides expression vectors, expression systems, and kits for the production of phototrophic cells that can be induced by small molecule modulators (e.g., inducers) to express an endo-hydrogenase and to harvest light energy for hydrogen production. By providing expression systems for inducible heterologous hydrogenases the present disclosure enables the production of hydrogen by phototrophic cells, such as cyanobacteria, that either lack endogenous, reversible hydrogenases or do not produce hydrogen under cell culture conditions that are optimal for the conversion of light energy into electrochemical energy, and ultimately optimal for hydrogen production.

The expression vectors, systems, or kits of this disclosure provide for hydrogenase genes controlled by regulatable promoters. Regulation occurs through transcription factors, such as transcriptional repressors or activators, which can interact with specific binding and transactivation sites on the regulatable promoters and can be regulated in their activity by small molecules or by temperature shifts. In some specific embodiments the vectors are monocistronic and code for the hydrogenase enzyme under control of the regulatable promoter, but do not encode the transcription factor. In other specific embodiments the vectors include expression cassettes for both the hydrogenase and the transcription factor. In other specific embodiments this disclosure provides a system that enables the expression of hydrogenase proteins and provides the hydrogenase under control of the regulatable promoter and the transcription factor on separate vectors. In other embodiments the abovementioned system is combined with a small molecule modulator to form a hydrogenase expression kit.

The transcription factors of this disclosure may be used in their wild-type forms or in mutant forms. Transcription factor mutations may include, without limitation, point mutations, insertions, or deletions. Mutations may activate or inhibit the activity of transcription factors, or change the nature of their activity, e.g. from transcriptional repressors into transcriptional activators, or change the temperature at which the transcription factors are optimally active. Alternatively, mutations may alter the interaction between transcription factors and small molecule modulators, e.g. by strengthening or weakening the interaction, or by turning small molecule activators into small molecule inhibitors or vice versa. Additionally, transcription factor fusion proteins may be used having altered functional properties than the wild-type form. Transcription factors may be used in their full-length form. Alternatively, transcription factor fragments, such as structural protein domains, including transactivation domains, or secondary structure elements may be used in isolation or as part of fusion proteins. In some embodiments of the disclosure the transcription factors are of prokaryotic origin and originate from organisms such as E. coli, S. typhimurium, R. melioti, E. cloacae, A. eutrophus, A. tumefaciens, or P. putida (see e.g., Henikoff et al., Proc. Natl. Acad. Sci. USA 85, 6602-06, 1988,). In other embodiments the transcription factors are of eukaryotic origin.

In one specific embodiment the transcription factor is a member of the LysR-family of prokaryotic transcriptional activators, such as NodD from Rhizobium meliloti. In a preferred embodiment the transcriptional activator is NahR from Pseudomonas putida. In other specific embodiments the transcription factors may include LeuO, CysB, LysR, IlvY, MetR, AmpR, AntO, and TfdO (see, e.g., Henikoff et al., Proc. Natl. Acad. Sci. USA 85, 6602-06, 1988). In other specific embodiments the transcription factor is the transcriptional repressor TetR. In specific embodiments the TetR repressor protein is fused to a transactivation domain, such as the transactivation domain of the Herpes Simples Virus protein VP16, and converted into a transcriptional activator (TetR-VP16). In specific embodiments small molecule modulators can activate TetR-VP16 or mutant versions of TetR-VP16 and thereby increase the expression of TetR-VP16-regulated genes. In other specific embodiments the transcription factor is a steroid hormone receptor, such as the estrogen receptor.

In one specific embodiment the regulatable promoter element includes the bidirectional nahR/nahG promoter region from Pseudomonas putida. Other regulatable promoter elements may include. In another specific embodiment the regulatable promoter element includes TetR response elements (TRE) or steroid hormone receptor response elements such as estrogen receptor response elements.

In some specific embodiments transcriptional repressors may, in the absence of small molecule modulators, repress the transcription of hydrogenase genes by 50%, 60%, 70%, 80%, 90%, 95%, 96%, 97%, 98%, 99%, or 100% compared to the level of gene transcription occurring in the absence of the respective transcriptional repressor. Through this repression of hydrogenase gene transcription the regulator protein can reduce expression of the hydrogenase protein by 50%, 60%, 70%, 80%, 90%, 95%, 96%, 97%, 98%, 99%, or 100% relative to the hydrogenase levels observed in the absence of the regulator protein. In some specific embodiments small molecule modulators may induce the transcriptional repressor to allow for 10%, 20%, 30%, 40%, 50%, 60%, 70%, 80%, 90%, or 100% of gene transcription occurring in the absence of the transcriptional repressor. Through this depression of hydrogenase gene transcription the small molecule modulator can induce 10%, 20%, 30%, 40%, 50%, 60%, 70%, 80%, 90%, or 100% of hydrogenase protein expression observed in the absence of the regulator protein. In some specific embodiments the regulator protein represses hydrogenase gene or protein expression below a level that is detectable by RT-PCR or western blot technologies. In these specific embodiments the small molecule modulator induces the regulator protein to allow at least for the expression of hydrogenase mRNA or protein levels that are detectable by RT-PCR or western blotting.

In some specific embodiments, in the absence of small molecule modulators, transcriptional activators promote low detectable levels of hydrogenase gene or protein expression. In these specific embodiments small molecule modulators may induce 2, 4, 6, 8, 10, 30, 100, 300, or 1.000-fold increases of hydrogenase protein levels or 10, 30, 100, 300, 1,000, 10,000, 30,000, 100,000, 300,000, or 1,000,000-fold increases in hydrogenase protein or mRNA relative to the expression levels occurring in the absence of small molecule modulators. In other specific embodiments, in the absence of small molecule modulators, transcriptional activators to not promote hydrogenase expression and hydrogenase mRNA or protein levels are undetectable by technologies such as RT-PCR or western blotting. In these specific embodiments the small molecule modulator induces the regulator protein to allow at least for the expression of hydrogenase mRNA or protein levels that are detectable by RT-PCR or western blotting.

In some specific embodiments the small molecule modulator is a gratuitous inducer of the transcription factor, i.e. a small molecule that cannot be metabolized in the host cell. In other specific embodiments the small molecule modulator is a metabolite made by the host cell or a small molecule that can be degraded by the host cell. In a preferred embodiment the small molecule modulator is salicylic acid. In other specific embodiments the small molecule modulator may include 2-aminobenzoate, tetracycline, and doxycyclin. In some specific embodiments the transcription factor mutants induce gene expression when shifted to lower temperatures than is optimal for the activity of the wild-type transcription factor. In other specific embodiments transcription factor mutants induce gene expression when shifted to higher temperatures than is optimal for the activity of the wild-type transcription factor. In some specific embodiments the temperature may be shifted by 3, 4, 5, 6, 7, 8, 9, or 10° C. up or down from the optimal wild-type temperature. In some specific embodiments the temperature shift may induce 2, 4, 6, 8, 10, 30, 100, 300, or 1.000-fold increases of hydrogenase protein levels or 10, 30, 100, 300, 1,000, 10,000, 30,000, 100,000, 300,000, or 1,000,000-fold increases in hydrogenase protein or mRNA relative to the expression levels occurring at the optimal wild-type temperature.

Hydrogenases of this disclosure utilize electrochemical energy stored across cellular membranes, such as the plasma membrane or other vesicular or organellar membranes, to power the endergonic production of hydrogen. In some embodiments, hydrogenases also utilize the electrochemical energy stored in form of proton gradients, i.e. the proton motive force, to mediate hydrogen production. In some embodiments, the hydrogenases may act as bidirectional catalysts and facilitate either the endergonic production or the exergonic consumption of hydrogen. In some embodiments the net activity of the bidirectional hydrogenase catalysts is determined by extracellular oxygen tension (see. e.g., Ng et al., PLoS One e4695, 2009). In preferred embodiments the proton gradients powering hydrogenase activities were established through the conversion of light energy. In other embodiments, the protein gradients were energized through the metabolism of organic matter, including organis molecules such as adenosinetriphosphate (ATP).

In some specific embodiments the hydrogenase enzyme is a group 4 hydrogenase. In some specific embodiments the hydrogenase is an endo-hydrogenase, i.e. a hydrogenase whose active site faces the cytoplasm rather than the extracellular space. Endo-hydrogenases may be integral-membrane proteins or soluble cytoplasmic proteins. Hydrogenases may be derived from organisms including aerobic, microaerophilic, or anaerobic bacteria, organotrophic bacteria or phototrophic bacteria, such as purple bacteria or cyanobacteria. In some phototrophic organisms, the expression of the endogenous endo-hydrogenase genes or proteins may be suppressed while photosynthesis is ongoing. Preferred endo-hydrogenases are integral-membrane, group-4, hydrogenases that are encoded by the seven-gene hyq cluster and share sequence homology with the respiratory complex I (NADH:quinone dehydrogenase) (Ng et al., PLoS One e4695, 2009). In some specific embodiments the endo-hydrogenase is the Hyq endo-hydrogenase from Azospirillum brasilense, Beijerinckia indica, Bradyrhizobium japonicum; Rhizobium leguminosarum bv. viciae, Rhodocista centenaria, Xanthobacter autotrophicus, or E. coli. In preferred embodiments the endo-hydrogenase is the Hyq endo-hydrogenase from Azorhizobium caulinodans, Rhodospirillum centenum (aka Rhodocista centenaria) SW or from Rhodopseudomonas palustris.

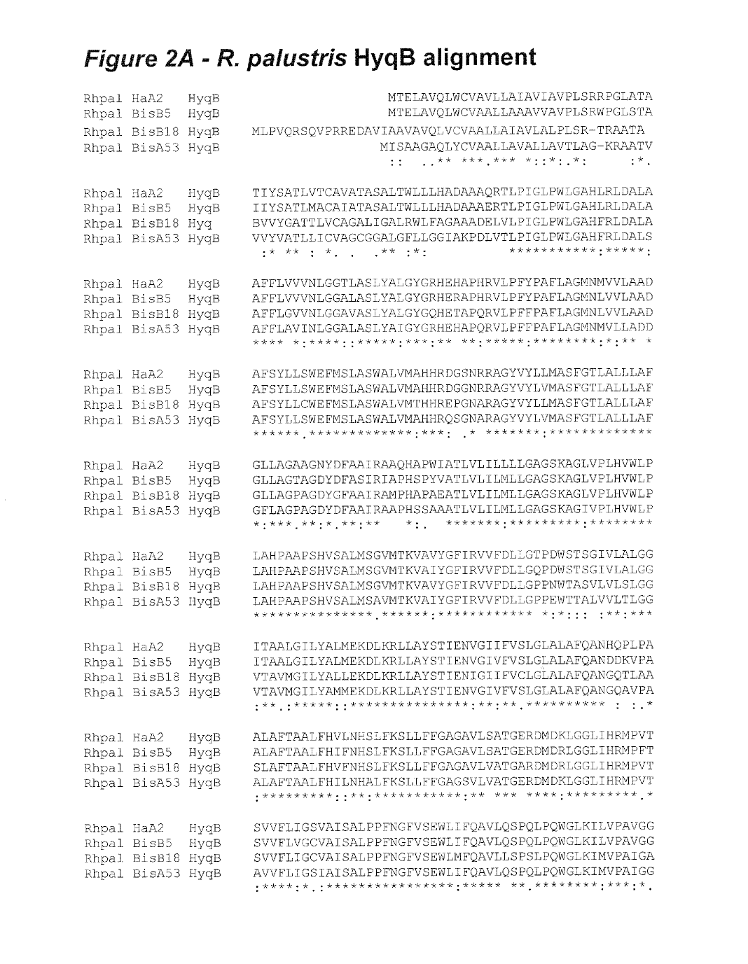

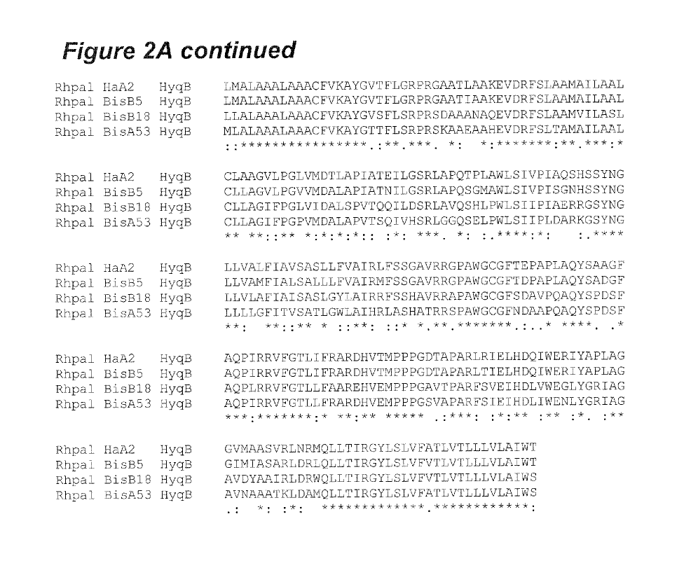

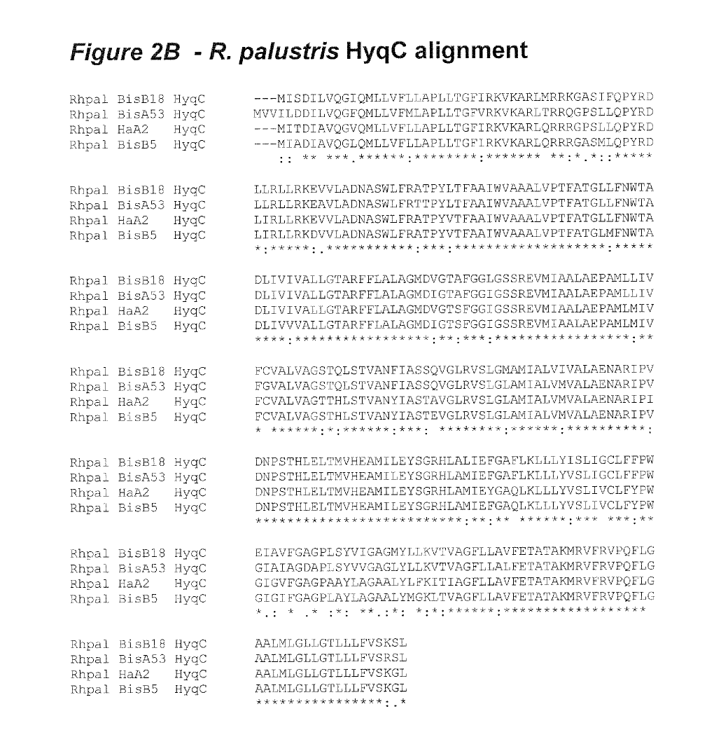

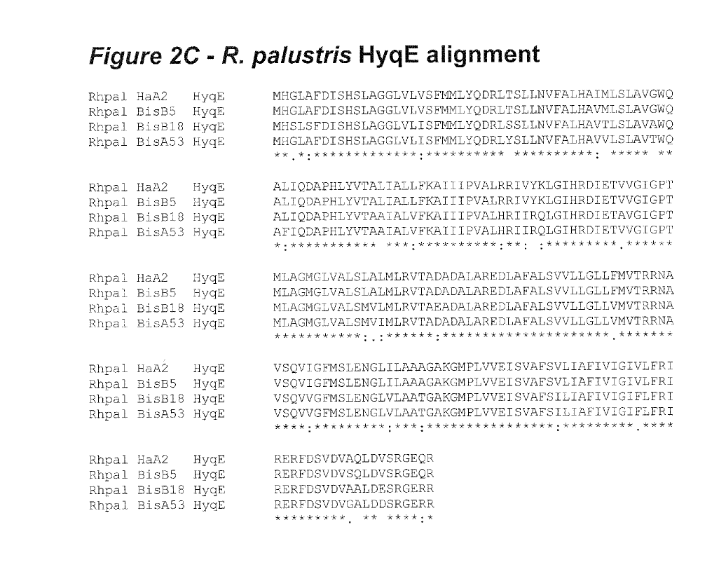

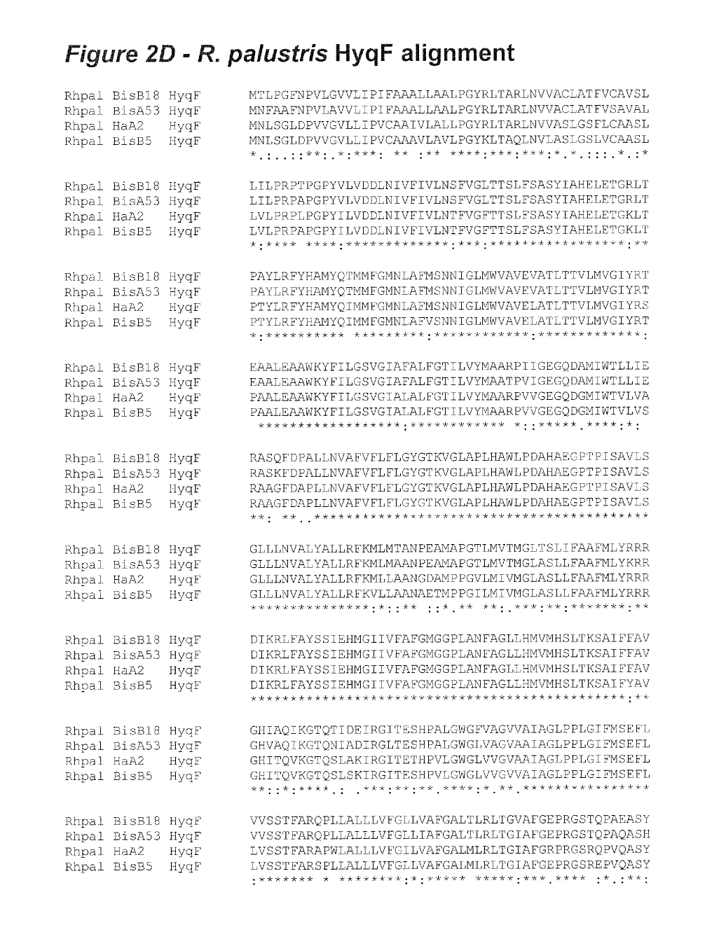

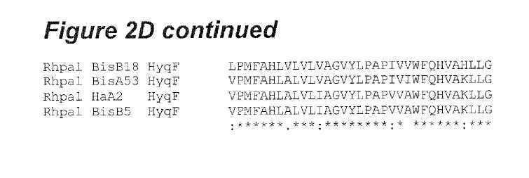

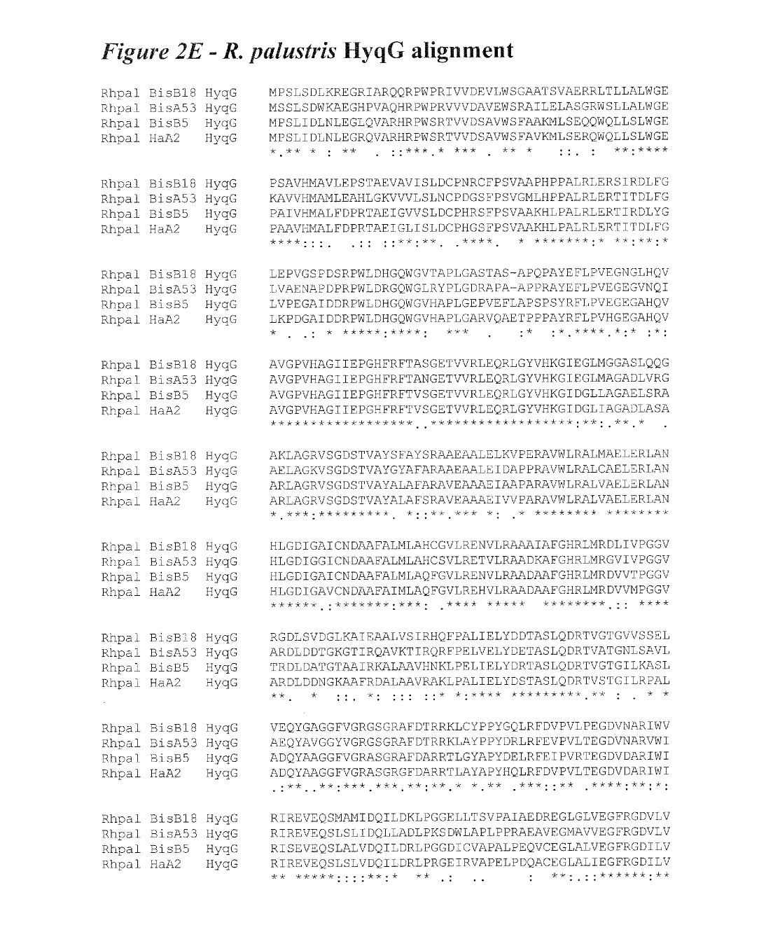

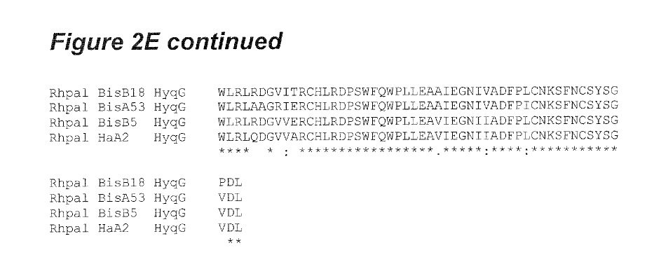

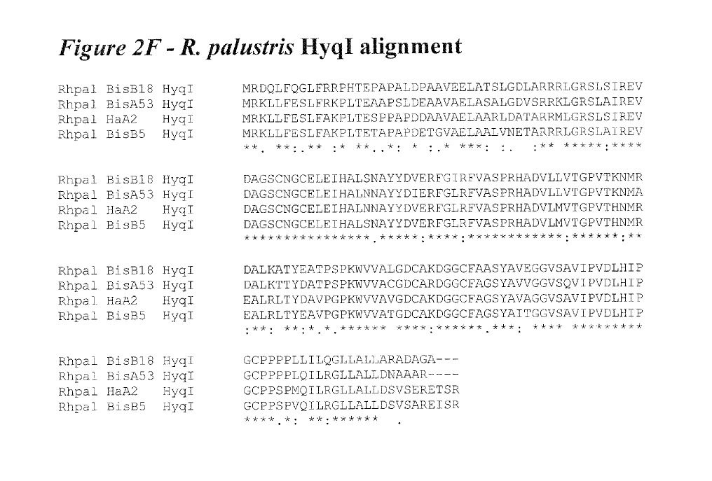

Clustal alignments of the amino acid sequences of the Hyq structural proteins of four wild type isolates of R. palustris are provided in FIG. 2. In some embodiments, the Hyq structural proteins of the present disclosure are at least 70%, 80%, 90%, 95%, 96%, 97%, 98%, 99%, or 100% identical to the Hyq structural proteins of an exemplary R. palustris strain (e.g., HaA2, BisB5, BisB18, or BisA53). In some preferred embodiments, the Hyq structural proteins of the present disclosure possess the residues conserved (e.g., residues shown with an asterix and/or a colon in FIG. 2) between exemplary R. palustris strains.

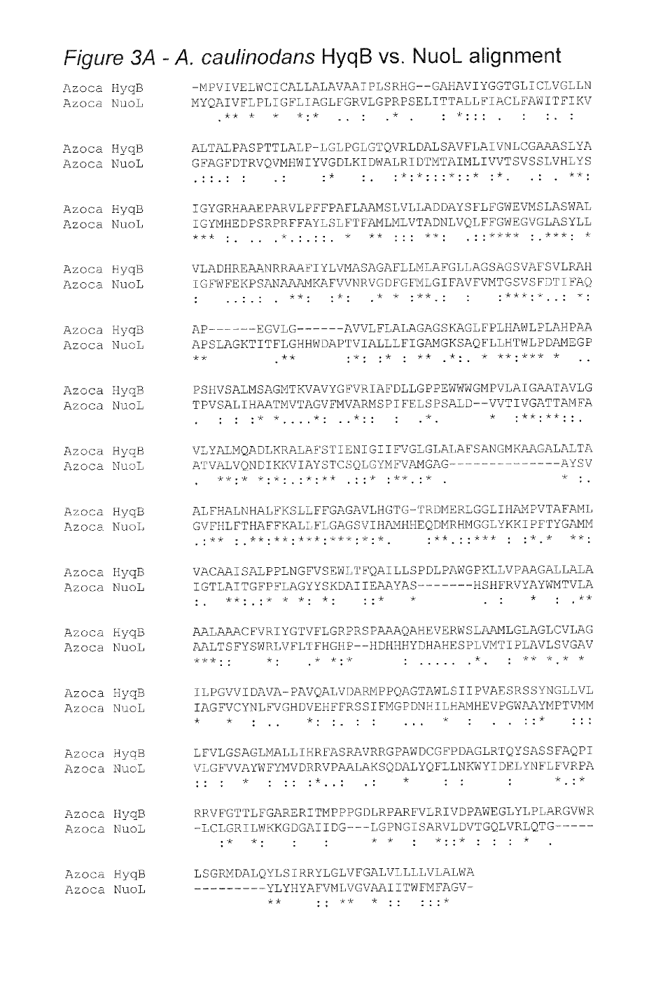

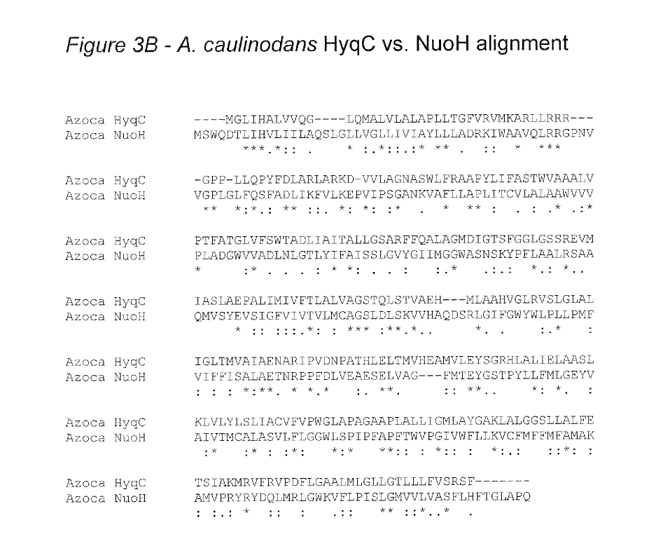

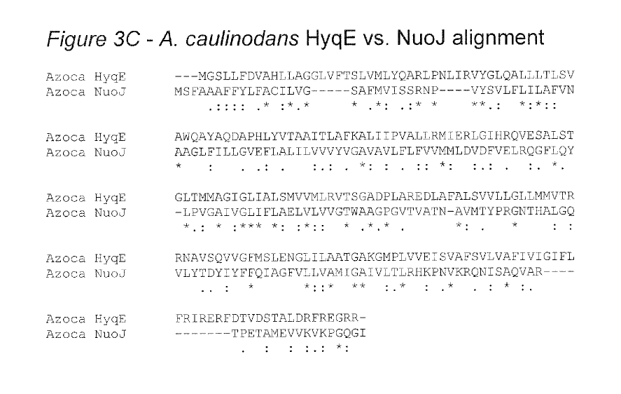

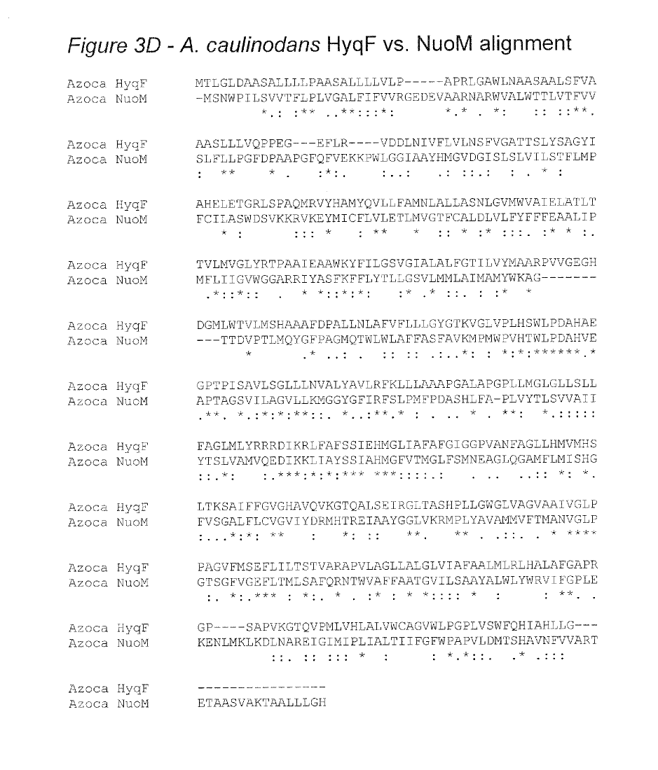

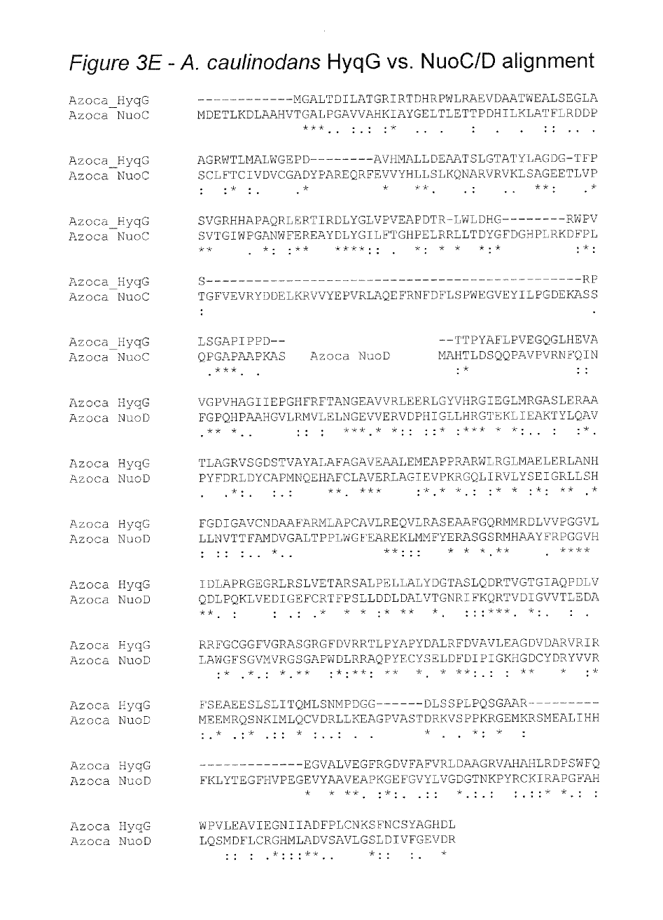

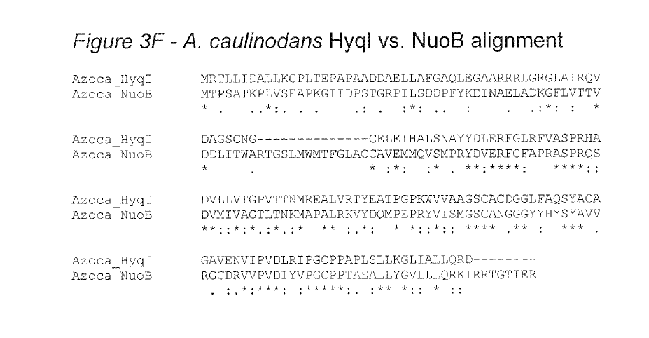

CLUSTAL alignments of the amino acid sequences of the Hyq structural proteins of A. caulinodans versus their Nuo (complex I) homologs are provided in FIG. 3. In some embodiments, the Hyq structural proteins of the present disclosure are at least 70%, 80%, 90%, 95%, 96%, 97%, 98%, 99%, or 100% identical to the HyqB structural proteins of an exemplary A. caulinodans strain. In some preferred embodiments, the Hyq structural proteins of the present disclosure possess the residues conserved (e.g., residues shown with an asterix and/or a colon in FIG. 3) between the Hyq structural proteins of A. caulinodans and their Nuo (complex I).

### Phototrophic Cells Capable of Inducible Hydrogen Production

In another embodiment the disclosure provides phototrophic cells that can be induced with small molecule modulators (e.g., inducers) to express hydrogenase enzymes and produce hydrogen gas. The hydrogenase enzymes are encoded by inducible hydrogenase genes. The hydrogenase genes may be endogenous or heterologous genes. Regardless of whether a given phototrophic cell contains endogenous hydrogenase genes, e.g. purple bacteria cells, or lacks endogenous hydrogenase genes, e.g. cyanobacteria cells, the cell may contain expression vectors or systems of this disclosure that allow for the inducible expression of heterologous hydrogenases. Alternatively, endogenous hydrogenase genes, such as the Hyq-family hydrogenase of R. palustris, may be modified in their promoter regions to include regulatable promoter elements that allow for inducible hydrogenase expression.

In some embodiments the phototrophic cells contain multiple expression vectors or systems of this disclosure. In other embodiments the phototrophic cells contain multiple endogenous hydrogenase genes that are controlled by regulatable promoters. In other embodiments the cells contain combinations of heterologous hydrogenase genes that were introduced using the expression vectors and systems of this disclosure and endogenous hydrogenase genes that are modified to include regulatable promoters. Where a cell contains multiple regulatable hydrogenase genes, these may be regulatable either in combination by the same transcription factor or individually by different transcription factors. Similarly, multiple regulatable hydrogenase genes may be inducible by either the same small molecule modulator or by different small molecule regulators.

The transcription factors interacting with the regulatable promoters of the hydrogenase genes may be either endogenous proteins of the host cell or heterologous proteins. Heterologous transcription factors may have been introduced into the host cell as part of an expression vector or system of the disclosure or independent of such a vector or system. Cells may contain a plurality of endogenous or heterologous transcription factors or a combination of heterologous or endogenous transcription factors. Where a cell contains a plurality of transcription factors that are capable of being induced by a small molecule modulator to promote the expression of hydrogenase genes or proteins this plurality of transcription factors may be promiscuous, i.e. inducible by the same small molecule modulator, or selective, i.e. inducible by specific small molecule modulators that activate at least one, but not all transcription factors.

The hydrogenase enzymes, regulatable promoters, and transcription factors contained in phototrophic cells of this disclosure are the same as those included in the expression vectors, systems, and kits of this disclosure.

In some embodiments the cells of this disclosure may further include inactivating mutations in genes encoding proteins whose activity detracts from the optimal hydrogen production by phototrophic cells in bioreactor settings. Such genes may include, without limitation, genes encoding hydrogen consuming enzymes, such as exo-hydrogenases or suppressors of hydrogenase expression, or, more generally, rate-limiting enzymes in non-essential energy intensive pathways. Alternatively, the cells may include inactivating mutations in genes degrading small molecule modulators. Inactivating mutations may include deletions, point mutations, insertions, or nonsense mutations.

Generally, cells of this disclosure include all cells capable of converting light energy into electrochemical energy. In some embodiments the cells convert light energy into proton gradients across membranes such as plasma membranes, vesicular membranes, or organellar membranes. In some embodiments the cells include light harvesting complexes (LHCs). The cells of this disclosure may include, without limitation, a broad spectrum of phototrophic prokaryotes and eukaryotes, such as purple bacteria, including Rhodopseudomonas (e.g. Rhodopseudomonas acidophila), Rhodospirillum (e.g. Rhodospirillum rubrum), Rhodobacter (e.g. Rhodobacter sphaeroides, Rhodobacter capsulatus), and Rhodovulum (e.g. Rhodovulum strictum, Rhodovulum adriaticum, and Rhodovulum sulfidophilum), cyanobacteria, including, Synechococcus spp., Synechococcus elongatus (e.g. PCC_{—}7942), and Synechocystis spp., and algae. Bacterial cells may be aerobic, microaerophilic, or anaerobic cells. Algea may include green, blue-green, and red algae, particularly those of Synechococcus sp. (e.g. PCC 7942), Chloroccales and Volvocales, and more particularly those of Chlamydomonas spp. (e.g. Chlamydomonas reinhardtii, Chlamydomonas moewusii, Chlamydomonas sp. stain MGA161, Chlamydomonas eugametos, Chlamydomonas segis), Chlorella (e.g. Chlorella vulgaris), Dunaliella (e.g. Dunaliella tetrolecta), Scenedesmus spp (e.g. Senedesmus obliguus), Anabaena (e.g. Anabaena variabilis ATCC 29413), Cyanothece (Cyanothece sp. ATCC 51142), and Anacystis (e.g. Anacystis nidulans). In preferred embodiments the cell is a Rhodopseudomonas palustris or Rhodospirillum centenum cell. In other preferred embodiments the cell is from the Rhodopseudomonas palustris strains HaA2, BisA53, Bis18, or BisB5.

In a preferred embodiment the cell is a R. palustris strain HaA2 or BisB5 cell in which the endogenous hyq+ promoter region was replaced with a gene-cassette encoding the Pseudomonas putida transcription factor NahR+, a transcriptional activator responsive to salicylic acid, and further including bidirectional nahR/nahG promoter regions to control the expression of NahR+ and the endogenous R. palustris Hyq endo-hydrogenase.

### Methods for the Production of Hydrogen Producers

In another embodiment the disclosure provides methods for the production of hydrogen producing cells. In some embodiments hydrogen producing cells can be produced by introducing expression vectors or systems of this disclosure into phototrophic cells. These embodiments enable the introduction of fully heterologous hydrogen production systems, including heterologous hydrogenases, regulatable promoters, and transcription factors into cells that may or may not contain the respective endogenous counterparts. Alternatively, these embodiments enable the introduction of partially heterologous hydrogen production systems, e.g. a heterologous hydrogenase under control of a heterologous regulatable promoter, into cells that may complement these heterologous components with endogenous components, e.g. endogenous transcription factors. In a specific embodiment a regulatable heterologous promoter and a transcription factor interacting with this promoter are introduced into a phototrophic cell to control the expression of an endogenous hydrogenase.

In specific embodiments the heterologous elements of the hydrogen production system, such as hydrogenase or transcription factor genes or regulatable promoters, may be introduced into the host cells using a single plasmid or vector or using a plurality of plasmids or vectors. If more than one heterologous element is introduced into the host cell, the different elements may be introduced simultaneously or in an iterative fashion. Techniques for introducing recombinant genetic material into the cell, techniques for directing the insertion of recombinant genetic material, such as regulatable promoters, into predefined genomic locations, and techniques for selecting successfully modified host cells vary with the host cell and are well known to a person of skill in the art of maintaining or manipulating the respective host cell (see, e.g., Coico et al., Current Protocols in Microbiology, Wiley, 2011; Link et al., J Bacteriol, 179:6228-6237, 1997). Generally, techniques required to produce phototrophic hydrogen producing cells include electroporation, chemical transformation, homologous recombination, and general molecular and cell biology techniques (Maniatis, Molecular cloning: A laboratory manual, Cold Spring Harbor Laboratory, 1982).

In a preferred embodiment the endogenous hyq+ promoter region in the R. palustris strains HaA2 or BisB5 is replaced with a gene-cassette encoding the Pseudomonas putida transcription factor NahR+, a transcriptional activator responsive to salicylic acid, and further including bidirectional nahR/nahG promoter regions to control the expression of NahR+ and the endogenous R. palustris Hyq endo-hydrogenase.

### Methods for Hydrogen Production Using Phototrophic Cells

In another embodiment the disclosure provides a method of producing hydrogen. The method includes maintaining phototrophic cells of this disclosure in culture, adding a small molecule modulator to the cell culture to induce the expression of a hydrogenase, expose the cell to light to provide the light energy required for hydrogen production, and allow for sufficient time for the cell to produce hydrogen. In specific embodiments the method may include the additional step of collecting the hydrogen produced by this method.

In some specific embodiments the cells may be maintained in either liquid suspension or in solid state cultures. Cells maintained in liquid suspension may be grown in a closed compartment, such as a bioreactor, that provides a controlled environment, or cells may be grown in an open compartment, such as a flask, tank, aquarium, pool, pond, lake, bay, or ocean. The cells may grow as individual cells or form celluar aggregates. The cells may also be grown in single cell layers or thick biofilms. Cells growing in biofilm aggregates may directly attach to solid surfaces or be embedded within self-produced matrices composed of extracellular polymeric substances or artificial matrices composed of synthetic polymers. Biofilm aggregated may be structured as thin, two-dimensional films or take on a three-dimensional structure by e.g. rolling up the two-dimensional biofilms into a carpet-like configuration or by providing external scaffolding to obtain multi-layer biofilm arrangements (see, e.g., U.S. Pat. No. 7,745,023).

In some specific embodiments the cell cultures are maintained in a cell culture environment such as a fermentor, bioreactor or photobioreactor. Bioreactors may operate as batch, fed batch, or continuous bioreactors, including continuous stirred-tank bioreactor models or chemostats. Bioreactors allow for the optimization of cell culture conditions to achieve optimal hydrogen production yields and process robustness. Environmental conditions that can be adjusted or monitored in a bioreactor include gas composition (e.g., air, oxygen, nitrogen, carbon dioxide), gas flow rates, temperature, pH, dissolved oxygen levels, and the agitation speed/circulation rate within the cell culture. Photobioreactors are bioreactors that incorporate a light source. Virtually any translucent container could be called a photobioreactor. General techniques known in the art for the large scale cell culture are known in the art, e.g., in Bailey and Ollis, Biochemical Engineering Fundamentals, McGraw-Hill (1986), and Shuler, Bioprocess Engineering: Basic Concepts, Prentice Hall (2001).

The cell culture process may be divided into a biomass expansion phase and a hydrogen production phase and different cell culture conditions, including nutrient compositions, illumination, temperature, and other conditions, may be chosen to optimize biomass expansion or hydrogen production. In some embodiments the cells will expand both during the biomass extension phase and during the hydrogen production phase. In other embodiments cell division will be arrested in the hydrogen production phase. Biomass expansion may occur under organotrophic or phototrophic conditions. Additionally, aerobic, anaerobic, or microaerophilic conditions may be chosen during biomass expansion. During the hydrogen production phase in photoautotrophic cell cultures, reducing agents, such as thiosulfate, sulfite, dimethyl sulfide, or trimethylamine, may be provided as part of the nutrient compositions to supply weak (low chemical potential/high electrical potential) electrons themselves incapable of H₂ production from hydrogen-ions donated by water. In some embodiments the cells of this disclosure are capable of conducting photoelectrolysis and derive the electrons needed for hydrogen production form water.

In some specific embodiments the small molecule modulator is added to the cell culture compartment at final concentrations sufficient to obtain the maximal induction of hydrogenase expression or the maximal production of hydrogen. In some specific embodiments the small molecule modulator is added at lesser concentrations, e.g. concentrations inducing half-maximal hydrogenase expression or hydrogen production. The addition of small molecule modulators to the cell culture may arrest further cell division and arrest the culture at a given density. Small molecule modulators may be added to the cell culture prior to illumination, after illumination, or simultaneously with illumination. In the event that multiple small modulators are used, such compounds may be added simultaneously, e.g. as a premixed composition, or separately at different time-points during the hydrogen production process.

The light used to induce and energize hydrogen production may be from natural sun light or an artificial light source, including a halogen lamp or a laser light source. The light may be applied in an unfiltered form or in a filtered form to only allow a preferred narrow band-width to illuminate the cell culture. In some embodiments preferred band-widths include 5 nm, 10 nm, 20 nm, 50 nm, or 100 nm. The light may be applied in different intensities, including high, medium, or low intensities. At high light intensities, optimal hydrogen yields are obtained after short periods of illumination, including 1, 5, 10, 15, 30, 45, or 60 min periods. At medium light intensities, approximately half-maximal hydrogen yields are obtained after short periods of illumination. Low light intensities are the minimal light intensities required to induce hydrogen production. In some embodiments cell cultures are illuminated with light continuously. In other embodiments cell cultures are intermittently illuminated with light and each light period is followed by a dark period. Light exposure may continue for a wide range of time periods, including 1 hr, 6 hrs, 12 hrs, 18 hrs, 1 day, 2 days, 3 days, 5 days, 1 week, 2 weeks, 3 weeks, 1 month, or longer. In one embodiment the light intensity is between 20 and 5000 μmol m⁻² sec⁻¹ (and all ranges within this range such as 100-3000, 1000-2000, 3500-5000, and so on) and illumination continues for up to 120 hours, but may be for a lesser period such as 24, 48, 64, or 96 hours.

In some embodiments hydrogen may be passively collected from the cell culture compartment, e.g. as it transitions by diffusion from the liquid or solid cell culture phase into the gas phase and the general environment surrounding the cell culture compartment. In other embodiments hydrogen may be extracted from the cell culture compartment, for example by sparging the cell culture compartment with air, nitrogen, carbon dioxide or, more generally, with an inert gas or mixtures of inert gases. In one embodiment the cell culture is sparged with a gas mixture of 0.1% O₂, 5% CO₂, balance N₂.

### DEFINITIONS

To facilitate an understanding of the embodiments disclosed herein, a number of terms and phrases are defined below.

Biofilm: A biofilm is an aggregate of microorganisms in which cells adhere to each other on a surface. Often aggregates are held together by extracellular material produced by the resident microorganisms.

Bioreactor: A bioreactor may refer to any manufactured or engineered device or system that supports a biologically active environment. For example, a bioreactor may be a vessel in which a biotechnological process is carried out which involves organisms or biochemically active substances derived from such organisms. This process can either be aerobic or anaerobic. These bioreactors may be cylindrical, ranging in size from liters to cubic meters, and made of stainless steel. A bioreactor may also refer to a device or system meant to grow cells or tissues in the context of cell culture. These devices are being developed for use in tissue engineering or biochemical engineering. Large scale immobilized cell bioreactors include moving media, also known as Moving Bed Biofilm Reactor (MBBR), packed bed, fibrous bed, or membrane bioreactors.

Endogenous nucleic acid: A nucleic acid that is a natural component of a cell's genome (e.g., a nucleic acid that is not introduced into the cell by laboratory methods). That is an endogenous nucleic acid is not a recombinant nucleic acid.

Heterologous nucleic acid: A nucleic acid that is not a natural component of a cell's genome (e.g., a nucleic acid that is introduced into the cell by laboratory methods). That is a heterologous nucleic acid is a recombinant nucleic acid. The sequence of the heterologous nucleic acid may be identical to the sequence of an endogenous nucleic acid of the cell. Alternatively, the sequence of the heterologous nucleic acid may be identical to the sequence of a different cell (e.g., same species or different species), or it may be a modified sequence, such as a codon-optimized sequence. The heterologous nucleic acid may be permanently inserted into the chromosomal DNA of the host cell, or it may be present transiently in the host cell (e.g., present in an episomal vector).

Illuminated conditions: Experimental conditions having sufficient light intensities for photosynthesis to occur.

Diazotorphic organism: A diazotroph is an organism that is able to grow without external sources of fixed nitrogen. Diazotrophs are bacteria and archaea that fix atmospheric nitrogen gas into a more usable form such as ammonia. Nitrogen fixation by diazotrophs is mediated by iron-molybdenum nitrogenase systems.

Organotrophic organism: An organotroph is an organism that obtains hydrogen or electrons from organic substrates.

Phototrophic organism: A phototroph is an organism that carries out photosynthesis to acquire energy. Phototrophs typically use solar energy to convert carbon dioxide and water into organic materials needed for biosynthesis and respiration. Some phototrophs are organotrophs, also known as photo-organotrophs.

Small molecule: A molecule having a molecular weight of 2,000 Da or less.

The use of the terms “a” and “an” and “the” and similar referents in the context of describing the disclosure (especially in the context of the following claims) are to be construed to cover both the singular and the plural, unless otherwise indicated herein or clearly contradicted by context.

Recitation of ranges of values herein are merely intended to serve as a shorthand method of referring individually to each separate value falling within the range, unless otherwise indicated herein, and each separate value is incorporated into the specification as if it were individually recited herein. Unless otherwise stated, all exact values provided herein are representative of corresponding approximate values (e.g., all exact exemplary values provided with respect to a particular factor or measurement can be considered to also provide a corresponding approximate measurement, modified by “about,” where appropriate).

### EXAMPLES

The present disclosure is described in further detail in the following examples, which are not in any way intended to limit the scope of the disclosure as claimed. The attached figures are meant to be considered as integral parts of the specification.

In the experimental disclosure which follows, the following abbreviations apply: M (molar); mM (millimolar); μM (micromolar); nM (nanomolar); pM (picomolar); mol (moles); mmol (millimoles); μmol (micromoles); nmol (nanomoles); pmol (picomoles); gm (grams); mg (milligrams); μg (micrograms); pg (picograms); L (liters); ml and mL (milliliters); μl and μL (microliters); cm (centimeters); mm (millimeters); μm (micrometers); nm (nanometers); U (units); V (volts); MW (molecular weight); sec (seconds); min(s) (minute/minutes); h(s) and hr(s) (hour/hours); ° C. (degrees Centigrade); QS (quantity sufficient); ND (not done); NA (not applicable); rpm (revolutions per minute); H₂O (water); dH₂O (deionized water); aa (amino acid); by (base pair); kb (kilobase pair); kD (kilodaltons); cDNA (copy or complementary DNA); DNA (deoxyribonucleic acid); ssDNA (single stranded DNA); dsDNA (double stranded DNA); dNTP (deoxyribonucleotide triphosphate); RNA (ribonucleic acid); OD (optical density); PCR (polymerase chain reaction); RT-PCR (reverse transcription PCR); DOT (Dissolved Oxygen Tension); and LHC (Light harvesting complex).

### Example 1

### Azorhizobium caulinodans Endo-Hydrogenase Hyq Generates Hydrogen In Vivo Under Low-Oxygen Conditions

This example demonstrates that the membrane-bound endo-hydrogenase Hyq from the diazotrophic microaerophile Azorhizobium caulinodans reverses its enzymatic activity in vivo in response to physiological O₂ availability. In optimized (20 μM DOT) diazotrophic liquid cultures, endo-hydrogenase serves as respiratory membrane e⁻ donor, consuming H₂ produced by Mo-dinitrogenase activity. In contrast, in microaerobic (≦1 μM DOT) cultures, this endo-hydrogenase serves as respiratory membrane terminal e⁻ acceptor, generating H₂.

Given its low (−420 mV) standard electrical potential, the biochemical hydrogen electrode normally serves as reductant in membrane e⁻ transfer processes, as hydrogen gas (H₂) is a strong e⁻ donor whereas H⁺ ions when combining are weak e⁻ acceptors. Yet among anaerobes carrying out mixed fermentations, H₂ production from H⁺ ions by membrane-bound hydrogenases employed as electron (e⁻) acceptor is quite common. Such membrane e⁻ transfer processes mitigate accumulation of toxins, such as carbon monoxide, and fermentative end-products such as formic and acetic acids which are volatilized and quickly dissipate, as gaseous mixtures of carbon dioxide (CO₂) and H₂. Presumably, these physiological benefits then compensate for the bioenergetic costs of H₂ production. By contrast, many aerobic bacteria employ membrane hydrogenases as respiratory e⁻ donor to O₂. Indeed, as aerobes need undertake oxidative phosphorylation, tightly coupling membrane e⁻ transfer and ATP resynthesis, membrane-bound hydrogenase activities are presumed operative only as e⁻ donor to e⁻ transfer chains. Yet, as determined during development of the present disclosure, the non-fermentative microaerophile A. caulinodans in pure microaerobic liquid cultures respires with both H⁺ ions and O₂ as terminal e⁻ acceptor, evolving H₂. Until now, the capacity of microaerophiles for hydrogenic respiratory e⁻ transfer has been masked as, in diazotrophic (using N₂ as sole N-source) culture, H₂ is also produced by Mo-dinitrogenase activity, a soluble process (Thorneley and Lowe, Molybdenum enzymes; Spiro, T. G., Ed.; Wiley-Interscience: New York, 221-84, Burgess and Lowe, Chem. Rev. 96:2983-11, 1996).

### Materials and Methods

Bacterial Strains and Media:

Azorhizobium caulinodans ORS571 wild-type (strain 57100), was originally isolated from Sesbania rostrata stem-nodules (Dreyfus and Dommergues, FEMS Microbiol. Lett. 10:313-17, 1981). Strain 61305R (Pauling et al., Microbiol. 147:2233-45, 2001), a 57100 derivative carrying an IS50R insertion in the (catabolic) nicotinate dehydrogenase structural gene served as ‘virtual’ wild-type for reported experiments; 61305R uses supplied nicotinate only as anabolic substrate for synthesis of pyridine nucleotides, for which 57100 is auxotrophic (Ludwig, J. Bacteriol. 165:304-07, 1986). Precise, in-frame deletion mutagenesis of A. caulinodans target genes was conducted out by ‘crossover PCR’ as previously described (Ng et al., PLoS ONE 4:e4695, 2009; and Link et al., J. Bacteriol. 179:6228-37, 1997).

Strain 66211R was constructed by gene conversion, as follows. Genomic DNA was isolated from strain 66035 nifD::Vi2021 (Donald, et al., J. Bacteriol. 165, 72-81, 1986) by standard techniques, sheared by repeated passage through an 18 gauge needle, purified by isopycnic centrifugation in 3M CsCl, and added to cell suspensions of strain 66204 (Ng et al., PLoS ONE 4, e4695, 2009) for electroporation. As A. caulinodans cells are quite fragile, cultures for electroporation were grown to saturation in defined medium with N-limitation under atmospheric (21%) O₂ to inhibit N₂ fixation, maintained at least 48 hr at 29° C. with constant agitation, iced, repeatedly pelleted and washed with sterile water on ice. Cells were resuspended (20 μl) in ice cold sterile 15% (v/v) glycerol, 1 μg purified, sheared genomic DNA was added, and cells were electroporated (τ=5 msec) at 14 kV cm⁻¹. Surviving bacteria were aerobically incubated 4 hr on defined medium to which (0.2% w/v) salt-free casamino acids and (0.1%) yeast extract were added and plated on defined medium (Donald et al., supra, 1986) supplemented with (10 mg l⁻¹) tetracycline (Tc). Resistant bacterial strains were analyzed for both nifD⁺ and nifD::Vi2021 alleles by PCR; most candidates proved nifD::Vi2021 haploids (Donald et al., supra, 1986) presumably arisen by gene conversion. The nifD::Vi2021 allele was resolved into a nifD::IS50R allele by screening stable Tc-sensitive derivatives (Loroch et al., J. Bacteriol. 177, 7210-21, 1995), yielding strain 66211R nifD::IS50R hupΔSL2 hyqΔRI7.

TABLE 1-1
Azorhizobium caulinodans Strains

| Stain | Genotype | Reference |
|---|---|---|
| 57100 | ORS571 wild-type | Dreyfus et al., FEMS Microbiol. Lett. 10: 313-17 |
| | | (1981) |
| 60035 | 57100 nifD35::Vi | Donald et al., J. Bacteriol. 165: 72-81 (1986) |
| 60035R | 60035 nifD35::IS50R | |
| 60107R | 57100 nifA107R | |
| 61305R | 57100 Nic⁻ 6-OH-NIC⁺ | Buckmiller et al., J. Bacteriol. 173: 2233-2245 (1991) |
| 66081 | 61305R hupΔSL2 | Ng et al., PLoS ONE 4, e4695 (2009) |
| 66081M | 66081 streptomycin- | |
| | resistant | |
| 66132 | 61305R hyqΔR/7 | Ng et al., PLoS ONE 4, e4695 (2009) |
| 66132P | 66132 spectinomycin- | |
| | resistant | |
| 66204 | 61305 hupΔSL2 hyqΔR/7 | Ng et al., PLoS ONE 4, e4695 (2009) |
| 66211R | 66204 nifD35 hupΔSL2 | |
| | hyqΔR/7 | |

Physiological Growth Measurements and Evolved H₂ Analysis:

Starter cultures of A. caulinodans strain 61305R and its derivatives (Buckmiller et al., J. Bacteriol. 173, 2233-2245, 1991) were aerobically cultured in minimal defined NIF liquid medium (Donald, et al., J. Bacteriol. 165, 72-81, 1986) supplemented with 0.3 mM ammonium (as a sole, limiting nitrogen source) and 1 μM nicotinate at 37° C. until growth arrested (cell densities ˜1×10⁸ cells ml⁻¹). For kinetic measurements of diazotrophy, arrested starter cultures were diluted one-hundred-fold in NIF medium (with 1 μM added nicotinate) into 30 ml serum vials, sealed with silicone rubber septa, sparged continuously (10 ml min⁻¹) with defined gas mixtures (e.g. 2% O₂, 5% CO₂, bal. N₂), and incubated at 29° C. At least three times per cell-doubling period, culture samples were removed, serially diluted, plated on rich GYPC medium (Donald et al., supra 1986), and incubated 48 hr at 37° C. Colonies were counted in triplicate.

To measure evolved H₂, sparge exhaust gas samples were analyzed by gas chromatography (Peak Laboratories LLC) using an HgO (reducing compound) photometer as detector (Vreman et al., J. Bacteriol. 179, 6228-37, 1997) and a fixed volume (25 μl) sampling loop. H₂ evolution rates were inferred from dilution rates of culture atmospheric volumes.

### Results

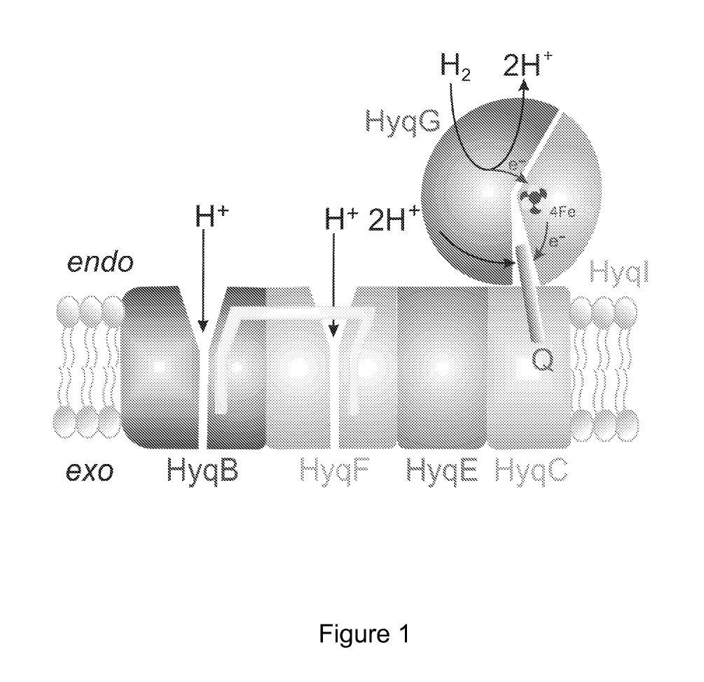

Hyq endo-hydrogenase is a simplified L-type respiratory complex I. The hyq operon of diazotrophic microaerophiles, represented here by Azorhizobium caulinodans (Entrez Gene Identifier: AZC_{—}4360-AZC_{—}4355) encodes an endo-hydrogenase including six structural proteins (Ng et al., PLoS ONE 4, e4695, 2009; and Ciccolella et al., PLoS ONE 5, e12094, 2010). In contrast, bacterial L-type NADH:quinone oxidoreductase (respiratory complex I) typically includes 14 proteins equally divided into membrane-integral (L₀) and membrane-peripheral (L₁) sub-complexes (Efremov et al., Nature 465, 441-45, 2010; Yagi and Matsumo-Yagi, Biochemistry 42, 2266-74, 2003; and Sazanov and Hinchcliffe, Science 311, 1430-36, 2006). The six structural A. caulinodans hyq genes all encode homologs of L-type respiratory complex I (Table 1-2). From superfamily analysis of a hidden Markov model library of protein structures (Gough et al., J. Mol. Biol. 313, 903-19, 2001), the HyqBCEF gene products were determined to represent membrane-integral (L₀) homologs. On the other hand, the HygGI proteins, which catalyze hydrogenase activity, were determined to be homologous to the three, core NuoCDB proteins of membrane-peripheral L₁. HyqG shows extensive homology to both group I hydrogenases and represents a fused NuoC::D (SSF56762). HyqI, a small FeS protein, is a NuoB (SSF56770) homolog with its conserved cys-55, cys-58 (Cys-X-X-Cys), cys-112 and cys-152 residues as likely coordinates of the N2 high-potential 4Fe4S center, which in complex I serves as immediate e⁻ donor to membrane quinone (Meinhardt et al., J. Biol. Chem. 262, 9147-53, 1987). The binding site for complex I membrane quinone as e− acceptor is a cavity formed between a four-helix bundle of NuoD, the H1 helix of NuoB, and transmembrane helix 1 of NuoH (Efremov et al., Nature 465, 441-45, 2010; and Yagi and Matsumo-Yagi, Biochemistry 42, 2266-74, 2003), all of which are conserved in the Hyq endo-hydrogenase complex (Table 1-2). By inference, the Hyq endo-hydrogenase of microaerophiles represents a simplified, core L-type H₂:quinone oxidoreductase coupled to a proton-motive pump (FIG. 1).

TABLE 1-2
A. caulinodans NADH:quinone oxidoreductase
(L-type respiratory complex I) and H2:quinone
oxidoreductase (Hyq endo-hydrogenase) structural homologs.

| | | | | |
|---|---|---|---|---|
| A. | EntrezGene | T. thermophiles | | EntrezGene |
| caulinodans | Identifier | complex I | A. caulinodans | Identifier |

L₁ subcomplex (membrane associated)

| | | | | |
|---|---|---|---|---|
| NuoB | AZC_1668 | Nqo6 | HyqI | AZC_4355 |
| NuoC | AZC_1669 | Nqo5 | HyqG(N-term.)^{†} | |
| NuoD | AZC_1670 | Nqo4 | HyqG(N-term.)^{‡} | AZC_4356 |
| NuoE | AZC_1671 | Nqo2 | | |
| NuoF | AZC_1672 | Nqo1 | | |
| NuoG | AZC_1674 | Nqo3 | | |
| NuoI | AZC_1676 | Nqo9 | | |

L₀ subcomplex (membrane-integral)

| | | | | |
|---|---|---|---|---|
| NuoA | AZC_1667 | Nqo7 | | |
| NuoH | AZC_1675 | Nqo8 | HyqC | AZC_4359 |
| NuoJ | AZC_1677 | Nqo10 | HyqE | AZC_4358 |
| NuoK | AZC_1678 | Nqo11 | | |
| NuoL | AZC_1679 | Nqo12 | HyqB | AZC_4360 |
| NuoM | AZC_1680 | Nqo13 | HyqF | AZC_4357 |
| NuoN | AZC_1667 | Nqo14 | | |

^{†}5′-end of hyqG encodes residues 40-124;
^{‡}3′-end of hyqG encodes residues 141-504

The nucleotide sequence of the Azorhizobium caulinodans hyq operon (Genbank Accession No. FJ378904) is set forth as SEQ ID NO:1:

| | |
|---|---|
| 1 | cagcatgagg cggctcagca gatagtcccc gcggaataac |
| | tggacgatgt tctggatgac |
| 61 | ggcgtcccgc atgaccggga agccggggtc gcctcgcaca |
| | tgggcggcga acgcggcgag |
| 121 | gtcgactggc gcagcccgga gctgaatgcg cccggcgtca |
| | aggccggcag ccggcagtcg |
| 181 | ctgggcgaga gcgttcatcc atgatccccg atcagcaggc |
| | gcgacgcacc agctaccccg |
| 241 | acgcaagggc catgccctag ccggtgcatc gacgatctca |
| | tcttatcatg gtgccgttgc |
| 301 | gcataaaccc tgatcccatc aatgggcatt gatgttcggg |
| | cgcctgttgg tcatgggcgc |
| 361 | gtatccaggg ttttattcac acgcatatca acgatagcgg |
| | gtgatgtcgc ggagcgtcaa |
| 421 | gtctgcgctc ctcatctttc gggaggtatg atggcgcgat |
| | gcgctcctct ggtgcaagcg |
| 481 | tcttgtcgcc ggacaggcgg agaaaaacgg gtcgatcccc |
| | atggccctgc ggacgctgcc |
| 541 | cctgcgtcct gcgaccgggc cggatatcgg aatgcatcgc |
| | gatcccgacc tttcatcgtg |
| 601 | cgagccgtgg cggcgcaccc gtgcggcctg tgcgtcgtca |
| | ttggatgatg tttaaaacat |
| 661 | cataatttta tgtttgatat tcactcctga cgtgattagg |
| | ctaattcatc aattctcagc |
| 721 | catgggtctt tcggccacat gatcaccgct gcgcaactgc |
| | gtgccgctcg cgccctgctc |
| 781 | gggctcgacc agaaggcgct ggccgcgctg tgcgcgctct |
| | ccgtgccgac catccagcgg |
| 841 | atggaggcca gtgacggggt cattcgcggc aatgtggatt |
| | ccctgatgaa gctcgtgggc |
| 901 | gcgctggagg gcgcgggcat ccagctcctc agcgagggca |
| | tggtgagtga agggggcggg |
| 961 | cgcggcgtgc gtctgcgcga ggcccccgtc cgccgcccgg |
| | cgccgcttcc ggacgaggcc |
| 1021 | tgacccatgc ctgtgatcgt cgagctctgg tgcatctgtg |
| | cgctgctggc tctggcggtc |
| 1081 | gcagccattc ccctgtcgcg ccatggcggt gcgcatgcgg |
| | tgatctatgg tggcacgggg |
| 1141 | ctcatctgcc tcgtgggcct gttgaacgct ctcacggccc |
| | tgccggccag ccccaccacg |
| 1201 | ctcgcgttgc cgctcggcct gccgggcctc ggcacgcagg |
| | tgcgcctgga tgcgctgagt |
| 1261 | gcggtcttcc tggccatcgt caatctgtgc ggcgcggctg |
| | ccagcctcta tgccatcggc |
| 1321 | tatggccgcc atgcggcgga gcccgcgcgg gtgctgccct |
| | tctttcccgc ctttcttgcc |
| 1381 | gccatgagcc tcgtgctgct ggcggatgac gcctacagct |
| | tcctgttcgg ctgggaggtg |
| 1441 | atgtcgctcg cctcctgggc gctggtgctg gcggatcatc |
| | gggaggccgc caaccggcgg |
| 1501 | gccgccttca tctatctcgt catggcgagc gccggcgcgt |
| | tcctgctgat gctcgctttc |
| 1561 | ggacttctgg cgggcagcgc ggggagcgtg gccttttccg |
| | tcctgcgcgc ccatgcgccg |
| 1621 | gagggtgtgc tgggtgccgt cgtgctgttt cttgcgctgg |
| | cgggtgccgg ctccaaggcg |
| 1681 | ggcctgtttc cgctgcatgc ctggctgccg ctcgcccatc |
| | ccgctgctcc cagccacgtc |
| 1741 | tcggcgctca tgagcgcggg catgaccaag gtggcggtct |
| | atggcttcgt gcgcatcgct |
| 1801 | tttgatctac tgggcccgcc cgaatggtgg tggggcatgc |
| | ccgtgctggc catcggcgcc |
| 1861 | gcgacggcgg tgctgggcgt gctctacgcg ctgatgcagg |
| | cggacctcaa gcgggcgctc |
| 1921 | gccttcagca ccatcgagaa catcggcatc atcttcgtcg |
| | ggctcggcct cgcgctcgcc |
| 1981 | ttctcggcca acggcatgaa agcggcgggg gcgctggccc |
| | tgacggcggc gctgttccac |
| 2041 | gcgctgaacc acgcgttgtt caagagcctt ctgttctttg |
| | gcgccggcgc ggtactgcac |
| 2101 | ggcacgggaa cacgggacat ggagcggctc ggtggcctta |
| | tccatgccat gcctgtgacg |
| 2161 | gccttcgcca tgctggtggc ctgcgcggcc atttcggcgc |
| | tgccgccgct gaacggcttc |
| 2221 | gtgtccgagt ggctcacctt ccaggccata ttgctgagcc |
| | cggacctgcc cgcctggggg |
| 2281 | ccgaagctgc tggtgccggc ggccggcgcc ctgcttgccc |
| | ttgccgcggc gctggcggcg |
| 2341 | gcctgcttcg tgcgcatcta cggcaccgtc ttcctcggcc |
| | gcccgcgctc gcccgctgcc |
| 2401 | gcgcaggccc atgaggtgga gcgctggtcg ctggccgcca |
| | tgctggggct ggccgggctg |
| 2461 | tgtgtgcttg ccggcatcct gccgggggtg gtgattgatg |
| | cggtggctcc cgccgtccag |
| 2521 | gcgctggtgg atgcgcgcat gcccccgcag gcgggcaccg |
| | cctggctttc catcattccc |
| 2581 | gtggccgaga gccgcagctc ctataacggg ctgctggtgc |
| | tcctctttgt gctcggctcg |
| 2641 | gctggcctga tggcgctgct catccatcgt ttcgcgtcgc |
| | gcgcggtgcg gcgcggcccg |
| 2701 | gcctgggact gcggcttccc agatgcaggc ctgcgcacgc |
| | agtatagcgc gtcgagcttc |
| 2761 | gcccagccca tccgccgcgt tttcggcacc accctgttcg |
| | gtgcgcgcga gcgcatcacc |
| 2821 | atgccgccgc cgggcgatct ccggccggcg cgtttcgtgc |
| | tgcgtatcgt cgatccagcg |
| 2881 | tgggaggggc tctatttgcc gctggcgcgg ggcgtgtggc |
| | gcctctcggg gcggatggac |
| 2941 | gccctgcaat atctctccat ccggcgctat ctcggcctcg |
| | tcttcggggc gctggtgctg |
| 3001 | ctgcttctgg tgctggcgct atgggcctga tccacgctct |
| | cgtcgtgcag ggcctccaga |
| 3061 | tggcccttgt gctcgcgctg gcgccgctgc tcaccggctt |
| | cgtgcgcgtg atgaaggccc |
| 3121 | gcctgctgcg ccggcgcggg ccgccgctgc tgcaacccta |
| | tttcgatctc gcgcgcctcg |
| 3181 | cccgtaagga tgtggtgctg gcgggcaatg cctcctggct |
| | gttccgagcc gcgccctatc |
| 3241 | tcatcttcgc ctccacctgg gtggcggcgg cattggtgcc |
| | cactttcgcc accggcctcg |
| 3301 | tcttcagttg gacggcggac ctcatcgcca tcacggcttt |
| | gctcggcagc gcccgtttct |
| 3361 | ttcaggcgct ggcgggcatg gacatcggca ccagttttcg |
| | cggcctcggc tccagccggg |
| 3421 | aggtgatgat cgcctcgctc gccgagcccg cactcatcat |
| | gatcgtctcc acgctggcgc |
| 3481 | tggtcgcggg ctcgacgcag ctctccaccg tcgccgaaca |
| | catgctggcg gcccatgtgg |
| 3541 | gcctgcgtgt ctcgttgggg ctagcactga ttggcctcac |
| | catggtggcc attgccgaga |
| 3601 | atgcgcgcat cccggtggac aatccggcga cccatctcga |
| | actcaccatg gtgcatgagg |
| 3661 | ctatggtgct ggaatattcc ggacgccatc tcgcgctcat |
| | cgagctcgcc gcatcgctga |
| 3721 | agctggtgct ctatctctcc ctcatcgcct gcgtgttcgt |
| | gccgtggggc cttgccccgg |
| 3781 | cgggcgcggc gccgctggcg ctcctcatcg gcatgctggc |
| | ctatggggcg aagctcgcgc |
| 3841 | tgggcggcag cctgctggcc ctgtttgaga cctccatcgc |
| | caagatgcgg gtcttccgtg |
| 3901 | tgccggattt cctcggcgcg gcgctgatgc tggggctgct |
| | cggcacgctg ctgctgttcg |
| 3961 | tgtcgcggag cttctgagca tgggcagcct tctcttcgat |
| | gtggcgcatc ttctggcggg |
| 4021 | cgggctcgtc ttcaccagcc tcgtgatgct ctatcaggcc |
| | cgcctgccga acctcatccg |
| 4081 | cgtctatggc cttcaggcgc tgctgctcac cctctccgtg |
| | gcctggcagg cctatgcaca |
| 4141 | ggatgcgccg catctctatg tgaccgccgc catcacgctc |
| | gccttcaagg cgctcatcat |
| 4201 | tcccgtggcg ctgctgcgga tgatcgagcg gctcggcatt |
| | caccggcagg tggaatccgc |
| 4261 | gctcagcacc ggcctcacca tgatggccgg catcggcctc |
| | attgccctgt ccatggtggt |
| 4321 | gatgctgcgc gtgacttccg gggccgatcc tctggcgcgg |
| | gaggacctcg ccttcgccct |
| 4381 | ttccgtcgtg ctgctgggcc tcctgatgat ggtgacgcgg |
| | cgcaacgccg tcagtcaggt |
| 4441 | ggtgggcttc atgtcgctgg agaacgggct gatcctcgcc |
| | gcgaccggtg ccaaggggat |
| 4501 | gccgctggtt gtggaaatct cggtcgcctt ctcggtgctg |
| | gtggccttca tcgtcatcgg |
| 4561 | catcttcctc ttccgcatcc gcgagcgctt cgacacggtg |
| | gattccacgg cgctcgaccg |
| 4621 | tttccgggag ggccgcagat gaccctcggc cttgatgcgg |
| | cttcggcctt gcttctgctg |
| 4681 | cccgccgcca gcgcgctcct gctgttggtg ctgccggcgc |
| | cccgcctcgg ggcatggctg |
| 4741 | aatgcggcga gcgccgcctt gtccttcgtc gcggcggcct |
| | cgctgctgct ggtgcagccc |
| 4801 | ccggagggag agttcctgcg ggtggacgat ctcaacatcg |
| | tctttctggt gctgaacagc |
| 4861 | ttcgtgggcg cgaccacgtc gctctacagc gccggctata |
| | tcgcccatga gctggagacg |
| 4921 | ggccgcctct cgcccgcgca gatgcgcgtc tatcacgcca |
| | tgtatcaggt gctgctgttc |
| 4981 | gccatgaatc tggcgctgct ggcgagcaat ctcggcgtga |
| | tgtgggtggc gatcgagttg |
| 5041 | gcgacgctca ccaccgtgct gatggtaggc ctctatcgca |
| | cgcccgccgc gatcgaggcg |
| 5101 | gcgtggaaat atttcatcct cggcagcgtc ggcatcgcgc |
| | tcgccttgtt cggcaccatc |
| 5161 | ctcgtctaca tggccgcccg gccagtggtg ggggaggggc |
| | acgacggcat gctctggacc |
| 5221 | gtgctcatga gccatgcggc ggccttcgat ccggcgctgc |
| | tcaacctcgc cttcgtcttc |
| 5281 | ctgctgctgg gctacggcac caaggtcggc ctcgtgccgc |
| | tgcactcctg gctgccggac |
| 5341 | gcccatgcgg aggggccgac gcccatttcc gccgtgctct |
| | cgggtctcct gctgaacgta |
| 5401 | gcgctctatg cggtgctgcg cttcaagctg ctgctggcgg |
| | ccgcgccggg cgcgctggcg |
| 5461 | ccggggccgc tgctcatggg gcttgggctg ctgtcgctgc |
| | tgttcgccgg gctgatgctc |
| 5521 | taccggcggc gggacatcaa gcgtctgttc gccttctcct |
| | ccatcgagca catggggctg |
| 5581 | atcgcgttcg cgttcggtat cggcgggccg gtggcgaatt |
| | tcgccggcct gctgcacatg |
| 5641 | gtgatgcata gccttaccaa gtccgccatc ttcttcggcg |
| | tcggccatgc ggtgcaggtg |
| 5701 | aagggcacgc aggcgctctc cgagatccgg ggcctcaccg |
| | ccagccatcc gctgctcggc |
| 5761 | tgggggctgg tggcgggcgt cgccgccatc gtcggcctgc |
| | cgccggcagg ggtgttcatg |
| 5821 | agcgaattcc tcattctcac ctcgaccgtt gcgcgggcgc |
| | ccgtgctggc ggggctgctg |
| 5881 | gcgctggggc tcgtcatcgc cttcgcggcc ctgatgctgc |
| | gcctgcatgc gctggccttc |
| 5941 | ggcgcgccgc gcgggccttc ggcaccggtg aagggcacgc |
| | aagtgcccat gctggtgcat |
| 6001 | ctggcgctcg tgtggtgcgc gggtgtgtgg ctgccggggc |
| | cgctggtaag ctggttccag |
| 6061 | cacatcgcgc atcttctggg ctgagggggc gagatgggcg |
| | cactcaccga tattctcgcc |
| 6121 | accggccgca tccgcaccga ccaccggccg tggctgcgcg |
| | ccgaagtgga cgccgcgaca |
| 6181 | tgggaggcgc tgtccgaggg gctggcggcg ggccgctgga |
| | cgctcatggc cctgtggggc |
| 6241 | gagcccgacg ccgtgcacat ggcgctgctg gatgaggccg |
| | cgacctcgct gggcacggcc |
| 6301 | acctatcttg cgggcgacgg cacgttcccc tccgttggcc |
| | gccaccatgc gccggcccaa |
| 6361 | aggctggagc gcaccattcg cgacctgtac ggcctggtgc |
| | cagtcgaagc gcccgatacg |
| 6421 | cgcctctggc tcgaccacgg ccgctggccg gtgtcgcgtc |
| | ctctcagtgg ggcccctatt |
| 6481 | ccgcccgaca cgaaccccta tgccttcctt cccgtcgatg |
| | ggcaagggct gcatgaagtg |
| 6541 | gcggtgggcc ccgtgcatgc gggcatcatc gagcctggcc |
| | atttccgctt caccgccaat |
| 6601 | ggcgaggccg tcgtgcggct ggaagagcgg ctcggctatg |
| | tccatcgcgg catcgagggg |
| 6661 | ctgatgcgcg gggcatccct ggagcgcgcc gccaccctcg |
| | ccggccgggt gtctggcgac |
| 6721 | agcacggtgg cctatgcctt ggccttcgcc ggcgccgttg |
| | aagccgccct cgagatggaa |
| 6781 | gcaccgccac gcgcccgctg gctgcggggc ctgatggcag |
| | agctggaacg cctcgccaat |
| 6841 | cacttcggtg acatcggtgc cgtctgcaac gacgctgcct |
| | ttgcgcggat gctcgccccc |
| 6901 | tgcgaggtgc tgcgcgaaca gatgctgcgt gcgagcgagg |
| | cggccttcgg ccagcgcatg |
| 6961 | atgcgcgacc tggtcgtccc cggtggcgtg ctgatcgatc |
| | tcgcgccgcg cggagagggc |
| 7021 | cggctgcgtt cgctggtgga aactgcgcgt tcggccctgc |
| | cggaactgct ggcgctttat |
| 7081 | gacggcaccg cctccttgca ggaccgcacc gtgggtacgg |
| | gcatcgcgca gccggatctg |
| 7141 | gtgcgccgct tcggttgcgg cggcttcgtg gggcgggcat |
| | cgggacgggg cttcgatgtc |
| 7201 | cggcggacgc taccttacgc tccttacgat gcgctgcgct |
| | tcgacgtggc ggtgctggag |
| 7261 | gcgggagatg tggacgcgcg ggtgcgcatc cgcttcagcg |
| | aggcggaaga gagcctttcg |
| 7321 | ctcattacgc agatgctcag caatatgccc gacgggggcg |
| | acctgtcttc gccgctgccg |
| 7381 | cagtccggcg cggcgcgcga gggcgtggca ctggtggaag |
| | ggttccgggg cgacgtcttc |
| 7441 | gccttcgtgc ggcttgatgc ggccgggcgc gtcgcccatg |
| | cccatctgcg cgatccctcc |
| 7501 | tggttccagt ggccggttct ggaggcggtc atcgaaggca |
| | atatcatcgc cgatttcccc |
| 7561 | ctctgcaaca aatccttcaa ctgctcctat gcggggcacg |
| | acctgtgagg cggcgcggat |
| 7621 | gcggacactc ctgatcgatg ccctgctgaa aggcccgctg |
| | accgagccgg cgccggcggc |
| 7681 | ggatgatgcg gagctgctgg ccttcggcgc gcagctcgaa |
| | ggggcggcgc ggcgcaggct |
| 7741 | cggccgcggt cttgccatcc ggcaggtgga tgcgggctcc |
| | tgcaacggct gcgaactcga |
| 7801 | aatccatgcc ctctccaacg cctattacga tctggagcgc |
| | ttcggtctgc gtttcgtcgc |
| 7861 | atcgccccgc catgcggacg tcctgctggt tacggggccg |
| | gtgacgacca acatgcggga |
| 7921 | ggcgctggtg cggacctatg aggcgacgcc cggcccgaaa |
| | tgggtggtcg cggccggaag |
| 7981 | ttgcgcctgt gacggtggcc tgttcgcgca gagctatgcc |
| | tgcgccgggg cggtggagaa |
| 8041 | tgtcattccg gtcgatctgc gtatccccgg ctgcccgcct |
| | gccccgctca gcctgctcaa |
| 8101 | gggcctgatc gcccttctgc aaagggatta gccaacacgt |
| | cccggtgcat tt |

The amino acid sequence of the A. caulinodans hyqR transcriptional activator, encoded by residues 739 to 1023 of the hyq operon, is set forth as SEQ ID NO:2:

MITAAQLRAARALLGLDQKALAALCALSVPTIQRMEASDGVIRGNVDSLM
KLVGALEGAGIQLLSEGMVSEGGGRGVRLREAPVRRPAPLPDEA.

The amino acid sequence of the A. caulinodans hyqB integral membrane protein, encoded by residues 1027 to 3030 of the hyq operon, is set forth as SEQ ID NO:3:

MPVIVELWCICALLALAVAAIPLSRHGGAHAVIYGGTGLICLVGLLNALT
ALPASPTTLALPLGLPGLGTQVRLDALSAVFLAIVNLCGAAASLYAIGYG
RHAAEPARVLPFFPAFLAAMSLVLLADDAYSFLFGWEVMSLASWALVLAD
HREAANRRAAFIYLVMASAGAFLLMLAFGLLAGSAGSVAFSVLRAHAPEG
VLGAVVLFLALAGAGSKAGLFPLHAWLPLAHPAAPSHVSALMSAGMTKVA
VYGFVRIAFDLLGPPEWWWGMPVLAIGAATAVLGVLYALMQADLKRALAF
STIENIGIIFVGLGLALAFSANGMKAAGALALTAALFHALNHALFKSLLF
FGAGAVLHGTGTRDMERLGGLIHAMPVTAFAMLVACAAISALPPLNGFVS
EWLTFQAILLSPDLPAWGPKLLVPAAGALLALAAALAAACFVRIYGTVFL
GRPRSPAAAQAHEVERWSLAAMLGLAGLCVLAGILPGVVIDAVAPAVQAL
VDARMPPQAGTAWLSIIPVAESRSSYNGLLVLLFVLGSAGLMALLIHRFA
SRAVRRGPAWDCGFPDAGLRTQYSASSFAQPIRRVFGTTLFGARERITMP
PPGDLRPARFVLRIVDPAWE GLYLPLARGVWRLSGRMDALQYLSIRRYL
GLVFGALVLLLLVLALWA.

The amino acid sequence of the A. caulinodans hyqC integral membrane protein, encoded by residues 3021 to 3977 of the hyq operon, is set forth as SEQ ID NO:4:

MGLIHALVVQGLQMALVLALAPLLTGFVRVMKARLLRRRGPPLLQPYFDL
ARLARKDVVLAGNASWLFRAAPYLIFASTWVAAALVPTFATGLVFSWTAD
LIAITALLGSARFFQALAGMDIGTSFRGLGSSREVMIASLAEPALIMIVS
TLALVAGSTQLSTVAEHMLAAHVGLRVSLGLALIGLTMVAIAENARIPVD
NPATHLELTMVHEAMVLEYSGRHLALIELAASLKLVLYLSLIACVFVPWG
LAPAGAAPLALLIGMLAYGAKLALGGSLLALFETSIAKMRVFRVPDFLGA
ALMLGLLGTLLLFVSRSF.

The amino acid sequence of the A. caulinodans hyqE integral membrane protein, encoded by residues 3980 to 4642 of the hyq operon, is set forth as SEQ ID NO:5:

MGSLLFDVAHLLAGGLVFTSLVMLYQARLPNLIRVYGLQALLLTLSVAWQ
AYAQDAPHLYVTAAITLAFKALIIPVALLRMIERLGIHRQVESALSTGLT
MMAGIGLIALSMVVMLRVTSGADPLAREDLAFALSVVLLGLLMMVTRRNA
VSQVVGFMSLENGLILAATGAKGMPLVVEISVAFSVLVAFIVIGIFLFRI
RERFDTVDSTALDRFREG.

The amino acid sequence of the A. caulinodans hyqF integral membrane protein, encoded by residues 4639 to 6084 of the hyq operon, is set forth as SEQ ID NO:6:

MTLGLDAASALLLLPAASALLLLVLPAPRLGAWLNAASAALSFVAAASLL
LVQPPEGEFLRVDDLNIVFLVLNSFVGATTSLYSAGYIAHELETGRLSPA
QMRVYHAMYQVLLFAMNLALLASNLGVMWVAIELATLTTVLMVGLYRTPA
AIEAAWKYFILGSVGIALALFGTILVYMAARPVVGEGHDGMLWTVLMSHA
AAFDPALLNLAFVFLLLGYGTKVGLVPLHSWLPDAHAEGPTPISAVLSGL
LLNVALYAVLRFKLLLAAAPGALAPGPLLMGLGLLSLLFAGLMLYRRRDI
KRLFAFSSIEHMGLIAFAFGIGGPVANFAGLLHMVMHSLTKSAIFFGVGH
AVQVKGTQALSEIRGLTASHPLLGWGLVAGVAAIVGLPPAGVFMSEFLIL
TSTVARAPVLAGLLALGLVIAFAALMLRLHALAFGAPRGPSAPVKGTQVP
MLVHLALVWCAGVWLPGPLVSWFQHIAHLLG.

The amino acid sequence of the A. caulinodans hyqG large (catalytic) endo-hydrogenase subunit, encoded by residues 6094 to 7608 of the hyq operon, is set forth as SEQ ID NO:7:

MGALTDILATGRIRTDHRPWLRAEVDAATWEALSEGLAAGRWTLMALWGE
PDAVHMALLDEAATSLGTATYLAGDGTFPSVGRHHAPAQRLERTIRDLYG
LVPVEAPDTRLWLDHGRWPVSRPLSGAPIPPDTNPYAFLPVDGQGLHEVA
VGPVHAGIIEPGHFRFTANGEAVVRLEERLGYVHRGIEGLMRGASLERAA
TLAGRVSGDSTVAYALAFAGAVEAALEMEAPPRARWLRGLMAELERLANH
FGDIGAVCNDAAFARMLAPCEVLREQMLRASEAAFGQRMMRDLVVPGGVL
IDLAPRGEGRLRSLVETARSALPELLALYDGTASLQDRTVGTGIAQPDLV
RRFGCGGFVGRASGRGFDVRRTLPYAPYDALRFDVAVLEAGDVDARVRIR
FSEAEESLSLITQMLSNMPDGGDLSSPLPQSGAAREGVALVEGFRGDVFA
FVRLDAAGRVAHAHLRDPSWFQWPVLEAVIEGNIIADFPLCNKSFNCSYA
GHDL.

The amino acid sequence of the A. caulinodans hyqI small endo-hydrogenase subunit, encoded by residues 7619 to 8131 of the hyq operon is set forth as SEQ ID NO:8:

MRTLLIDALLKGPLTEPAPAADDAELLAFGAQLEGAARRRLGRGLAIRQV
DAGSCNGCELEIHALSNAYYDLERFGLRFVASPRHADVLLVTGPVTTNMR
EALVRTYEATPGPKWVVAAGSCACDGGLFAQSYACAGAVENVIPVDLRIP
GCPPAPLSLLKGLIALLQRD.

The Genbank GENE identifiers for additional hyq operon sequences are RC1 1420-1415 (R. centenum SW), RPD 3855-3850 (R. palustris, BisB5), and RPB 1260-1265 (R. palustris, HaA2). A. caulinodans and R. palustris HyqG proteins are 68% identical and 85% conserved (typical numbers for pairwise comparisons of the six Hyq structural proteins).

In Growth-Optimized Diazotrophic Liquid Cultures Held at 20 μM DOT, Endo-Hydrogenase Activity Serves In Vivo as Respiratory Membrane e⁻ Donor in Uptake of Endogenous H₂.

As described above, A. caulinodans possesses both respiratory exo- and endo-hydrogenases, for which null mutants in unlinked hyq (endo-hydrogenase) and hupSL (exo-hydrogenase) structural genes were isolated. In growth-optimized liquid diazotrophic cultures open to the environment, exo-hydrogenase mutants grow normally, whereas endo-hydrogenase mutants grow slowly (Ng et al., PLoS ONE 4, e4695, 2009), possibly reflecting net loss to the environment of H₂ produced by Mo-dinitrogenase activity (Thorneley and Lowe, Molybdenum Enzymes, Spiro, T. G., Ed; Wiley-Interscience: New York, 221-284, 1985; and Burgess and Lowe, Chem. Rev. 96, 2983-11, 1996). To test this model, H₂ evolution rates of such liquid batch cultures under continuous sparge were measured. A. caulinodans strains were cultured diazotrophically under 2% O₂, 5% CO₂, bal. N₂ sparge 72 at 29° C., which yields optimum N₂-dependent growth rates. DOT in these sparged cultures held steady in the range of 18-20 μM O₂, as measured potentiometrically with a Clark polarographic-type electrode (Thermo-Orion 97-08. Culture exit gas streams were then periodically sampled and analyzed for evolved H₂ by gas chromatography using as detector a mercuric oxide reducing-compound photometer (Peak Laboratories, RCP1). In these diazotrophic cultures, both endo-hydrogenase mutant 66132 and hyq, hupSL (exo-, endo-hydrogenase) doubl e⁻ mutant 66204 showed ten-fold elevated H₂ evolution rates relative to both hyq+, hup+ 80 parent 61305R and hupSL exo-hydrogenase mutant 66081 (Table 1-3B). Because hyq single mutants showed higher H₂ evolution rates when compared to hupSL single mutants, endo-hydrogenase activity then seems disproportionately responsible for recycling endogenous H₂ produced by Mo-dinitrogenase activity.

When defined media were supplemented with 1 mM L-glutamine, measurable H₂ evolution by all strains was negligible. In A. caulinodans cultures, such L-glutamine sufficiency yields complete repression of the nif regulon, including nifD and nifK genes encoding Mo-dinitrogenase (Donald et al., J. Bacteriol. 165, 72-81, 1986). In A. caulinodans ORS571 wild-type, nitrate (AZC_{—}0679) and nitrite (AZC_{—}0680-AZC_{—}0682) reductases are soluble and assimilatory; neither nitrate nor nitric oxide serves as respiratory e⁻ acceptor. Likewise, nifD hyq hupSL triple-null mutant 66211R, cultured with similar defined medium supplemented with 1 mM nitrate, evolved H₂ at baseline levels in comparison with exo-, endo-hydrogenase double-mutant 66204 (Table 1-3A). Therefore, physiological H₂ evolution was entirely due to Mo-dinitrogenase activity. Sparge rates for these enclosed liquid cultures were standardized to allow culture atmosphere exhaust rates of 0.5 min⁻¹. For hyq single mutants, endogenous H₂ might have dissipated sufficiently fast to be inefficiently recycled by exo-hydrogenase activity, presumably as a diffusion-controlled process, given culture densities of some 1×10⁸ cells ml⁻¹.

Under Microaerobic Conditions (≦1 μM DOT), In Vivo Endo-Hydrogenase Activity Reverses and Operates as Respiratory Electron Acceptor, Supplementing Limited O₂, and Continuously Evolving H₂.

Similar diazotrophic liquid batch cultures were established for 24 hr, allowing cell densities to reach approximately 1×10⁸ ml⁻¹, then switched to a microaerobic sparge gas (0.1% O₂, 5% CO₂, bal. N₂). Thereupon, DOT levels, measured potentiometrically, declined steadily. When 1 μM DOT as upper threshold culture was breached, true microaerobic physiology (DOT insufficient to sustain conventional cytochrome aa₃ oxidase activity) was established (Kaminski et al., J. Bacteriol. 178, 5989-94, 1996; and Ludwig, Res. Microbiol., 155, 61-70, 2004). Growth rates of diazotrophic, microaerobic cultures were periodically sampled and viable cell counts determined; for experimental purposes, all cultures then maintained exponential growth for 72+ hr, with measured cell doubling-times of 30±1.5 hr at 29° C.

Methyl viologen (1,1′-dimethyl-4,4′-bipyridinium) and methylene blue (3,7-bis[dimethylamino]-phenothiazin-5-ium), both serve as alternative e⁻ acceptors for NADH:quinone oxidoreductase (respiratory complex I) and, when reduced, as e⁻ donors to cytc-dependent cytochrome oxidases, bypassing cytochrome bc₁ (respiratory complex III) activity, substantially uncoupling oxidative phosphorylation (Scott and Hunter, J. Biol. Chem. 241, 1060-66, 1966). Accordingly, these compounds were deployed to microaerobic cultures as in vivo respiratory e⁻ transfer probes. At experimentally sampled time points, anoxic methylene blue solution (2 μM final) was injected into duplicate microaerobic cultures, which invariably turned and remained visibly blue to unaided eyesight. If sparges were then withdrawn and resulting sealed cultures were incubated with agitation at 29° C.; within 60 min, all cultures turned completely colorless (anoxic). Therefore, all growing microaerobic cultures retained respiratory activity, and employed sparges continuously provided O₂ at rates exceeding those of physiological O₂ consumption.

In such microaerobic cultures, H₂ evolution rates dramatically increased. For parental 61305R cultures, H₂ evolution rates increased more than fifty-fold; for exo-hydrogenase mutant 66081, H₂ evolution rates increased almost four thousand-fold and persisted for 72+ hr. Yet, for endo-hydrogenase mutant 66132, H₂ evolution rates increased only fifteen-fold (Table 1-3C). By comparison, when both nifA null mutant 60107R and nifD hyq hupSL triple-null mutant 66211R were cultured microaerobically with 1 mM nitrate added as N-source, H₂ evolution was <3% that of exo-hydrogenase mutant 66081 (Table 1-3D). Therefore, microaerobic H₂ evolution was largely owed to activities associated with and transcriptionally controlled by the N₂ fixation regulon (Loroch et al., J. Bacteriol. 177, 7210-21, 1995). When 4 mM methyl viologen was added to microaerobic exo-hydrogenase mutant 66081 cultures, within five minutes, H₂ evolution had subsided to levels similar to those of untreated microaerobic exo-, endo-hydrogenase mutant 66204 cultures (Table 1-3C; and data not presented). Therefore, the bulk of the H₂ evolved at extraordinarily high rates by microaerobic cultures of exo-hydrogenase mutant 66081 was owed to respiratory membrane endo-hydrogenase activity. That so, in vivo endo-hydrogenase activity then reverses in response to physiological O₂ availability. In optimized diazotrophic cultures (20 μM DOT), endo-hydrogenase primarily operates in H₂ uptake mode, consuming endogenous H₂ as a respiratory e⁻ donor. In microaerobic (≦1 μM DOT) cultures, endo-hydrogenase shifts to H₂ production mode, using H⁺ ions as a respiratory e⁻ acceptor. Given (72+ hr) sustained, elevated rates of respiratory membrane H₂ production by these organotrophic cultures, net reductant needs to be quantitatively generated by oxidizable substrates (e.g., succinate, L-malate were provided).

Biological Relevance of Simultaneous H₂ Production and H₂ Uptake in A. caulinodans Under Microaerobic Cell Culture Conditions.

Without wishing to be bound by theory the following functional model is proposed. A. caulinodans is typical of obligate oxidative (non-fermentative) bacteria whose organotrophic culture taps NADH (Eₒ′=−320 mV) as predominant in vivo respiratory e⁻ donor. Generally, NADH dependent hydrogenic respiration (Eₒ′=−420 mV) cannot be appreciably exergonic as a steady state cellular respiratory process and is presumably unable to drive oxidative phosphorylation. To ascertain whether exo-hydrogenase was beneficial and/or whether endo-hydrogenase was detrimental to A. caulinodans microaerobic growth in diazotrophic microaerobic cultures, a physiological H₂ cross-feeding experiment was conducted with (1:1 cell:cell) mixed cultures of spontaneous streptomycin-resistant exo-hydrogenase mutant 66081 and spectinomycin-resistant endo-hydrogenase mutant 66132 derivatives. In mixed microaerobic co-cultures, neither differences nor changes in growth rates were observed when compared to pure cultures of single strains (data not presented). Whereas, in growth optimized (20 μM DOT), endo-hydrogenase mutants doubled every 8.8 hr, whereas exo-hydrogenase mutants doubled every 7.2 hr (Ng et al., PLoS ONE 4, e4695, 2009). As any microaerobic H₂ cross-feeding in mixed cultures had no effect on growth rates, endo-hydrogenase dependent hydrogenic membrane e⁻ transfer was not then detrimental to (i.e., posed a disproportionate metabolic load on) growth rate. Neither did exo-hydrogenase activity, using exogenous H₂ as respiratory e⁻ donor, then benefit growth rate beyond that offered by endogenous NADH as substrate for oxidative phosphorylation. By inference, O₂ availability was growth-limiting for all diazotrophic microaerobic cultures. However, for sparged microaerobic cultures provided succinate as sole organotrophic substrate, yields were distinct. Total cell counts for endo-hydrogenase mutants were reproducibly at least 30% greater than those of exo-hydrogenase mutants. Whereas, cell yields of optimized (20 μM DOT) diazotrophic, sparged liquid cultures were relatively less for endo-hydrogenase mutants (Ng et al., supra, 2009). Therefore, endo-hydrogenase mediated microaerobic respiratory e⁻ transfer, and resulting H₂ evolution, significantly uncouples oxidative phosphorylation and lowers ATP yields of net exogenous substrate oxidations.

In liquid culture experiments all bacteria were exposed to similar physiological environments. In natural habitats, microaerophilic bacteria grow three dimensionally as colonies or biofilms, and cell physiology must vary positionally. Internal cells, experiencing relative nutrient and O₂ limitation, potentially preferentially undertake some hydrogenic respiration. Evolved H₂ then diffuses to superficial cells, whose higher nutrient and DOT levels facilitate oxidative phosphorylation and cell growth, with H₂ employed as an added energy source. Were such a physiological division of labor positional among these bacteria, H₂ cross feeding would augment growth of the pure colony as a whole.

TABLE 1-3
H₂ evolution by A. caulinodans diazotrophic cultures

| | | | |
|---|---|---|---|
| A. caulinodans | Genotype | H₂ evolved† | relative H₂ evolved‡ |

A. N₂ and NO₃⁻ as N-sources (20 μM DOT)

| | | | |
|---|---|---|---|
| 66204 | Δhyq ΔhupSL | 460 | 46.0 ± 5.0 |
| 66211R | nifD Δhyq ΔhupSL | 10 | 1.0 ± 0.2 |

B. N₂ as sole N-source (20 μM DOT; optimized)

| | | | |
|---|---|---|---|
| 61305R | nif+ hyq+ hup+ | 12 | 1.0 ± 0.2 |
| 66081 | ΔhupSL | 16 | 1.3 ± 0.3 |
| 66132 | Δhyq | 175 | 15.0 ± 1.6 |
| 66204 | Δhyq ΔhupSL | 540 | 45.0 ± 5.0 |

C. N₂ as sole N-source (≦1 μM DOT; microaerobic)

| | | | |
|---|---|---|---|
| 61305R | nif+ hyq+ hup+ | 7,100 | 1.0 ± 0.2 |
| 66081 | ΔhupSL | 61,000 | 9.0 ± 1.0 |
| 66132 | Δhyq | 2,600 | 0.4 ± 0.04 |
| 66204 | Δhyq ΔhupSL | 14,000 | 2.2 ± 0.4 |

D. N₂ and NO₃⁻ as N-sources (≦1 μM DOT; microaerobic)

| | | | |
|---|---|---|---|
| 60107R | nifA | 1,100 | 1.0 ± 0.2 |
| 66211R | nifD Δhyq ΔhupSL | 1,600 | 1.5 ± 0.3 |

Structural and Mechanistic Model for H₂ Production and H₂ Uptake by Hyp Superfamily Endo-Hydrogenases.

Hyq is a respiratory endo-hydrogenase, present in a diverse group of diazotrophic microaerophiles but not diazotrophic aerobes such as Azotobacter spp., the latter adapted for modern atmospheric O₂ levels. As a microaerophile, A. caulinodans employs two ultrahigh O₂ affinity cytochrome oxidase activities, cytbd and cytcbb₃, allowing microaerobic cultures to drive oxidative phosphorylation and remain oxidative at nanomolar DOT (Kaminski et al., J. Bacteriol. 178, 5989-94, 1996). Thus, diazotrophic microaerophiles sustain oxidative phosphorylation at submicromolar DOT (Ludwig, Res. Microbiol. 155, 61-70, 2004). As determined during development of the present disclosure, this membrane-bound endo-hydrogenase reverses in vivo in response to physiological O₂ availability. In optimized (20 μM DOT) diazotrophic liquid cultures, endo-hydrogenase serves as a respiratory membrane e− donor, consuming H₂ produced by Mo-dinitrogenase activity. In microaerobic (≦1 μM DOT) cultures, this endo-hydrogenase serves as a respiratory membrane terminal e− acceptor, evolving H₂.

This respiratory endo-hydrogenase is encoded as a hyq operon including 6+ orthologous genes. The six, inferred Hyq proteins, all homologs of bacterial respiratory complex I (14 proteins), presumably constitute a simplified, core L-type H₂:quinone oxidoreductase activity. Inferred L₁ (membrane-peripheral) proteins include HyqG (fused NuoDC homolog) and HyqI (NuoB homolog) and Lₒ (membrane-integral) proteins include HyqC (NuoH), HyqE (NuoJ), HyqB (NuoL) and HyqF (NuoM). L₀ transmembrane H⁺ pumping, which builds and sustains respiratory membrane proton-motive force, is bio-mechanically coupled to L₁ electrochemical oxidation of NADH at the expense of membrane quinone (Efremov et al., Nature 465, 441-45, 2010). Given structural homology and functional analogy with respiratory complex I, whose membrane-peripheral L₁ sub-complex faces the cell cytoplasm, this H₂:quinone oxidoreductase is termed an endo-hydrogenase. In A. caulinodans, from all evidence, membrane-integral endo-hydrogenase activity is specifically employed in diazotrophic cultures and in symbiotic legume nodules fixing N₂ (Ciccolella et al., PLoS ONE 5, e12094, 2010). This Hyq endo-hydrogenase is phylogenetically classified as a group IV hydrogenase. All six Hyq proteins are homologs of both respiratory complex I proteins and Ni,Fe-type group I hydrogenases are widely distributed among aerobic bacteria (Vignais and Billoud, Chem. Rev. 107, 4206-72, 2007). Previously, group IV hydrogenases were considered H₂ evolving, in association with strictly fermentative metabolism and thus limited to anaerobic bacteria (Böhm, et al., Mol. Microbiol. 4, 231-43, 1990; Fox et al., J. Bacteriol. 178, 1515-24, 1996; Andrews et al., Microbiol. 143, 3633-47, 1997; and Fox et al., J. Bacteriol. 178, 6200-08, 1996). As reversible group IV hydrogenases operating in H2 uptake mode presumably also possess a chemiosmotic workload by analogy to respiratory complex I (FIG. 1), H₂ uptake is relatively slow by comparison with group I hydrogenases (Vignais and Billoud, Chem. Rev. 107, 4206-72, 2007).

Among eight genera (A. caulinodans, Azospirillum brasilense, Beijerinckia indica, Bradyrhizobium japonicum; Rhizobium leguminosarum bv. viciae, Rhodopseudomonas palustris, Rhodocista centenaria, Xanthobacter autotrophicus) of microaerophiles carrying orthologous hyq operons, the HyqG superfamily (SSF56762) includes group I hydrogenase catalytic proteins whose Ni,Fe binuclear active site is coordinated by four, conserved Cys residues, of which two bridge the catalytic Ni and Fe═C═O center (Volbeda et al., J. Am. Chem. Soc. 118, 12989-96, 1996). However, the HyqG family lacks both group I hydrogenase N-terminal and C-terminal Cys-X-X-Cys motifs. In contrast, three Cys residues (A. caulinodans Cys-258, Cys-491, and Cys-497) are completely conserved in the HyqG family. Correspondingly, neither complex I Nqo5 nor Nqo4 superfamily members carry a Ni,Fe-binuclear center.

The binding site for respiratory complex I membrane quinone, its ultimate e⁻ acceptor, is a cavity formed between a four-helix bundle of NuoD, the H1 helix of NuoB, and transmembrane helix 1 of NuoH (Efremov et al., Nature 465, 441-45, 2010; and Yagi and Matsumo-Yagi, Biochemistry 42, 2266-74, 2003), all of which are conserved in the Hyq endo-hydrogenase. As Hyq endo-hydrogenase activity is membrane-integral and, as with respiratory complex I, presumably uses membrane quinone as ultimate e⁻ acceptor, H₂ production in microaerobic cultures represents hydrogenic membrane e⁻ transfer. As added membrane quinone competitors such as methyl viologen strongly and promptly inhibit A. caulinodans hydrogenic membrane e⁻ transfer, membrane quinone likely participates as an e⁻ carrier. As NADH then serves as an ultimate e⁻ donor, the chemically reduced state of intracellular NAD pools allows quantitative inferences on the bioenergetics of hydrogenic membrane e⁻ transfer. When A. caulinodans cultures are shifted from aerobiosis to microaerobiosis, intracellular NAD pools change from <1% reduced to 40% reduced (Pauling et al., Microbiol. 147, 2233-45, 2001). The latter value poises the intracellular NAD half-cell bio-electrochemical potential at approximately −315 mV for respiratory complex I activity. Though the standard hydrogen electrode midpoint potential is −420 mV, with hydronium ion at unit activity (10⁻⁷M) as H⁺ donor, H₂ may then be produced in vivo up to pH₂=1.6 kPa under standard conditions, assuming no involved chemiosmotic work. Indeed, under physiological conditions for H₂ evolution by exo-hydrogenase mutants, pH₂ values in sparge exit gases approach 1 kPa 251 in A. caulinodans microaerobic cultures open to the environment.

While, H⁺ ions presumably serve as respiratory membrane e⁻ acceptor of last recourse, H₂ production by cellular NADH as respiratory membrane e⁻ donor may allow some oxidative phosphorylation, considered as an equilibrium process. In accord with standard models (Efremov et al., Nature 465, 441-45, 2010), as a vector membrane process, respiratory complex I activity may be written:

NADH+Q+4H⁺_{N}→NAD⁺+QH₂+3H⁺_{P} [1]

where N denotes the electronegative (endo) face, P the electropositive (exo) face of the respiratory membrane, and Q membrane quinone. By analogy, endo-hydrogenase activity, carrying two of the three (complex I) H⁺-pumping L₀ subunits (FIG. 1) may be written:

H₂+Q+2H⁺_{N} [symbol] QH₂+2H⁺_{P} [2]

Assuming endo-hydrogenase activity fully reversible, we may write NADH-driven hydrogenic respiration as eqs. [1]-[2]:

NADH+2H⁺_{N}→NAD⁺+H₂(↑)+H⁺_{P} [3]

Thus, NADH driven hydrogenic membrane e⁻ transfer might do chemiosmotic work (contribute to proton-motive force), provided evolved H2 were to dissipate (↑). As a biochemical standard process coupled to oxidative phosphorylation, ATP yield would then be <15% of NADH driven respiration at the expense of O₂ as terminal e⁻ acceptor (˜P:H₂<0.5). Indeed, respiratory H₂ evolution is substantive, as for diazotrophic microaerobic cultures open to the environment, biomass yields of exo-hydrogenase mutants are notably diminished, presumably reflecting net H₂ loss to the environment.

Hydrogenic membrane e⁻ transfer by A. caulinodans exo-hydrogenase mutants in diazotrophic microaerobic liquid batch cultures persists at high rates for 72+ hr. In microaerobic cultures, sum totals of evolved H₂ (2e⁻ reduction) at 72 hr are 230±30 μmol per 10⁹ cells, representing oxidation of some 25% of total (340 μmol) succinate supplied to these cultures as sole organotrophic energy source and quantitatively converted to poly-β-hydroxbutyrate (5.5 e⁻ oxidation) as an organic end-product (Pauling et al., Microbiol. 147, 2233-45, 2001). At the same time, A. caulinodans growth in diazotrophic, microaerobic cultures is quite slow but steady (t_{D}=30 hr). Thus, such microaerobic cultures, given excess organotrophic substrate, e.g. succinate, and submicromolar DOT must simultaneously respire using both O₂ and H+ ions as terminal e⁻ acceptor at similar rates (within the same order of magnitude).

In A. caulinodans, endo-hydrogenase activity is strictly correlated with diazotrophy and is bidirectional, whereas exo-hydrogenase activity is unidirectional and is also important for chemoautotrophy with exogenous H₂ as energy source. Therefore, the two, respiratory hydrogenases possess distinct physiological roles (Ng et al., PLoS ONE 4, e4695, 2009). Among fermentative anaerobes, hydrogenic membrane e⁻ transfer is well described (Böhm, et al., Mol. Microbiol. 4, 231-43, 1990; Fox et al., J. Bacteriol. 178, 1515-24, 1996; Andrews et al., Microbiol. 143, 3633-47, 1997; and Fox et al., J. Bacteriol. 178, 6200-08, 1996). Whereas, for obligate aerobes such as A. caulinodans, which must run oxidative phosphorylation, hydrogenic membrane e⁻ transfer as a sustained physiological process seems paradoxical, as it significantly uncouples oxidative phosphorylation, hydrogenic membrane e⁻ transfer seems counterproductive to growth and sustenance of aerobic, non-fermentative bacteria.

### Example 2

### Direct Coupling of Photoenergy Conversion to In Vivo Hydrogen Production by Purple Photosynthetic Bacteria

Briefly, Rhodospeudomonas palustris are purple, non-sulfur microaerophile bacteria that grow both phototrophically (on light energy) and organotrophically (on organic-chemical energy). This example describes the genetical engineering of R. palustris to eliminate hyq+ operon repression when shifted to phototrophic culture. Specifically, recombinant R. palustris cells described in this example are capable of harvesting light energy to power the endogenic process of hydrogen biosynthesis.

Among R. palustris wild-isolates, the presence of the hyq+ operon encoding endo-hydrogenase activity, as well as the 20-gene hup+ operon encoding exo-hydrogenase activity, is quite variable. Bioinformatic analyses showed that R. palustris wild-isolates possess either one, or both operons, and that all are single-copy. Typically, both R. palustris hyq+ and hup+ operons are expressed during diazotrophic (N₂ fixing), organotrophic liquid culture in three wild-isolate type-strains: HaA2 hyq+, CGA010 hup+, and BisB5 hyq+, hup+. However, the hyq+ operon is repressed in both HaA2 and BisB5 strains when physiologically shifted to phototrophic culture (i.e., darkness to light).

To strongly express the hyq+ operon in R. palustris phototrophic cultures, using both in vitro and in vivo genetic engineering techniques, the wild-type hyq+ promoter (cis-acting control of gene expression) regions in strains HaA2 and BisB5 were replaced by in vivo homologous recombination. In its place, a 1.4 kbp DNA fragment gene-cassette comprising Pseudomonas putida nahR+ was introduced encoding a transcriptional repressor/activator protein responsive to salicylic acid, and flanking, bidirectional nahR/nahG promoter (P) regions. True haploid, perfect gene replacement (ΔPhyq::nahR+) derivatives of both R. palustris HaA2 and BisB5 were confirmed and verified by PCR-based nucleotide sequencing analyses.

The ˜1.4 kb Pseudomonas putida nahR+ cassette conferring salicyclic acid responsiveness to an operatively-linked (downstream) hyq operon fragment (Phyq) is set forth as SEQ ID NO:9

| | |
|---|---|
| 1 | ggaacggtgt cctgtctttc atggggctcg ttcactgcta |
| | acttacctgc ttccctgtct |
| 61 | gtcaattctc tctatcctgc gatcccgcga agaaccaaaa |
| | aagctcgaca gagggcgcgg |
| 121 | tcattttagg tcgggcggat cggcgccgcc ggctcggctg |
| | gtgtgccgca cagcaccgcc |
| 181 | tacgtgagct gccagttgat gaacttcccc cgttgccagc |
| | tagggcgcaa gcgggctgta |
| 241 | taagatcact gcccatcaca ttgatcggct cggatttttt |
| | ctcaatccgt aaacaggtca |
| 301 | aacatcggtt gcctgcaacc aaatattggc taggtccttg |
| | tggtaccttc gcatgccaga |
| 361 | acatgttgat ggctatttca ggcaagacga ctgggtgcgg |
| | caaggcgctt aggccgaagg |
| 421 | gctccacgca gcagtcggct aaacgtatcg gcacagtggc |
| | gagcagatcg gtgcgctgga |
| 481 | ggatgtggcc aacggcggcg aagtgcggca cttccagacg |
| | gatgtcgcgc cggatgccga |
| 541 | cccgtgtcat gtacgtgtcc acctcgccgt ggccggtgcc |
| | agcggcgatg acacgcacgt |
| 601 | ggccgtagga acagaagcgc tccagagtca ggggttcgcg |
| | ggtgactgga tggtccttgc |
| 661 | gacataggca cacgtagtga ttctggagca gccggcgctg |
| | aaagaagcca gtttgcagat |
| 721 | tgggaagcag gcccacggcc aagtccacgg ttccgttctg |
| | caaggcctgc atcaggctca |
| 781 | tcgaactgtc gcgcaccgta ctgatcacgc aattgggggc |
| | ctggtgagcc agcacatcca |
| 841 | tcagccgcgg catgaagtag atctcgccaa tgtcggtcat |
| | ggccagggtg aaggtacgct |
| 901 | cgctggtcag cggatcgaag ctttcatggt gctgtagggc |
| | gttgcgcagt gcgtgcatgg |
| 961 | ccgaagtgac gggctcggcc agatgcgcgg catagggtgt |
| | gggttccatt ccctgatgtg |
| 1021 | tgcgcacgaa gagtgggtcc tgtagcgagg tgcgcaggcg |
| | tttcagcgca ttgctcacgg |
| 1081 | caggctgggt caggcccagg ttctccgcag tgatagagac |
| | gcgtctgtcg accagcaact |
| 1141 | ggttgaacac caccagcagg tttaaatcca ggtcacgcag |
| | ttccatgggg cctcgcttgg |
| 1201 | gttattgctg gtgcccggcc gggcgcaata ttcatgttga |
| | tgatttatta tatatcgagt |
| 1261 | ggtgtattta tcaatattgt ttgctccgtt atcgttatta |
| | acaagtcatc aataaagcca |
| 1321 | tcacga. |

Both HaA2 and BisB5 ΔPhyq::nahR derivatives could be phototrophically cultured as wild-type in defined liquid media in the absence of added salicylic acid. When late-exponential microaerobic, phototrophic liquid batch cultures were supplemented with (1 μM) salicylic acid, (3 mM) thiosulfate, the hyq+ operon was strongly derepressed. Further culture growth as measured by viable cell counts abruptly ceased. However, when exit gas streams of these sparged cultures were resampled, H₂ was now evident and at remarkably high levels. When the employed light source was extinguished, H₂-levels in culture exit gases immediately declined. Additionally, these salicylate-induced phototrophic cultures retained no, detectable ATP resynthesis capability.

In conclusion, these engineered R. palustris ΔPhyq::nahR strains, when induced in phototrophic cultures, quantitatively operate hydrogenic photoelectron transport. Thus, they directly couple light energy conversion to electrochemical energy, continuously output as H₂, approaching unit efficiency.

### Example 3

### In Vivo Hydrogen Production by Purple Photosynthetic Bacteria Using Photoenergy Conversion on an Electrode Surface

Graphite electrodes were previously shown to serve as efficient electron donors for anaerobic respiratory bacteria, such as Geobacter sulfurreducens (Gregory et al., Environ, Microbiol, 6:596-604, 2004). Recombinant R. palustris ΔPhyq::nahR strains, cultured and photoinduced in the absence of extrinsic electron-acceptors (e.g., organic-C sources, thiosulfate, carbon monoxide, dimethyl sulfide as per Example 2) were applied to graphite electrode surfaces as thin film aqueous-based latex emulsions (Gosse et al., Biotechnol. Prog. 23:124-130, 2007) infused with magnetite nanoparticles (termed “photobacterial paints”), which conduct electrons to bacterial surface pili (Kato et al., Proc. Natl. Acad. Sci. USA 109:10042-10046, 2012). When these “painted” graphite electrodes were charged with a weak electrical potential just sufficient to reduce the photobacterial periplasmic cytochrome c₂ pool (+660 mV absolute; −4.7 eV relative to electrons at rest in vacuo) and then illuminated under oxygen gas restrictive atmospheres as detailed in Example 2, hydrogen gas was again produced continuously. Supplied with this low electrical charge, photobacterial hydrogen gas yields were entirely light-dependent and quantitatively similar to those detailed above. Thus, the recombinant, phototrophic bacteria of the present disclosure, given light energy and an electric current carried through a conductive material to photobacterial cell-surfaces, sustainably produce hydrogen as coupled photochemical process.

1. A recombinant, phototrophic bacterium comprising:
a) a nucleic acid encoding structural proteins of an endo-hydrogenase operon;
b) a heterologous nucleic acid encoding a transcriptional repressor/activator protein responsive to an inducer; and
c) a heterologous nucleic acid comprising bidirectional promoter regions; wherein the heterologous nucleic acid comprising bidirectional promoter regions is located in operable combination with the nucleic acid encoding the structural proteins on one side and the heterologous nucleic acid encoding the transcriptional repressor/activator protein on another side, and wherein the transcriptional repressor/activator induces expression of the structural proteins when the recombinant bacterium is cultured in the presence of the inducer.

2. The recombinant bacterium of claim 1, wherein the nucleic acid encoding the structural proteins of the endo-hydrogenase operon is endogenous to the bacterium.

3. The recombinant bacterium of claim 1, wherein the bacterium is an alpha-proteobacterium.

4. The recombinant bacterium of claim 3, wherein the alpha-proteobacterium is Rhizobiales bacterium.

5. The recombinant bacterium of claim 3, wherein the alpha-proteobacterium is a Rhodopseudomonas palustris strain in which the endo-hydrogenase operon is a hyq operon that is repressed when the alpha-proteobacterium is shifted to phototrophic culture.

6. The recombinant bacterium of claim 1, wherein the nucleic acid encoding the structural proteins of the endo-hydrogenase operon is heterologous to the bacterium.

7. The recombinant bacterium of claim 1, wherein the structural proteins of the endo-hydrogenase operon are at least 70% identical to Rhodopseudomonas palustris structural proteins of SEQ ID NOS:10-15.

8. The recombinant bacterium of claim 7, wherein the bacterium is an alpha-proteobacterium selected from the group consisting of Rhodopseudomonas palustris, Rhodospirillum centenum, and Azorhizobium caulinodans.

9. The recombinant bacterium of claim 7, wherein the bacterium is a cyanobacterium selected from the group consisting of a Synechococcus sp. and a Synechocystis sp.

10. The recombinant bacterium of claim 9, wherein the nucleic acid encoding the structural proteins of the endo-hydrogenase operon is codon-optimized for expression in the cyanobacteria.

11. The recombinant bacterium of claim 1, wherein the structural proteins of the endo-hydrogenase operon comprise HyqB, HyqC, HyqE, HyqF, HyqG and HyqI.

12. A method for producing hydrogen photosynthetically, the method comprising:
a) growing the recombinant bacterium of claim 1 phototrophically in media in the absence of the inducer to produce a bacterial culture;
b) supplementing the media of the bacterial culture with the inducer; and
c) exposing the bacterial culture with light in the presence of the inducer so as to express the structural proteins of the endo-hydrogenase operon, thereby producing H₂ gas photosynthetically from the bacterial culture.

13. The method of claim 12, wherein the bacterial culture produced in step a) is a late-exponential phase bacterial culture.

14. The method of claim 12, wherein steps a) through c) take place under microaerobic conditions.

15. The method of claim 14, wherein the microaerobic conditions comprise sparging the bacterial culture with nitrogen.

16. The method of claim 15, further comprising step d) extracting the H₂ gas from gas streams from the nitrogen-sparged bacterial cell culture.

17. The method of claim 12, wherein step b) further comprises supplementing the media with one or more of the group consisting of thiosulfate, sulfite, dimethyl sulfide, and trimethylamine.

18. The method of claim 12, wherein step c) further comprises subjecting the bacterial culture to an electric current.

19. The method of claim 12, wherein step c) further comprises preparing an emulsion comprising magnetite nanoparticles, latex and bacteria from the bacterial culture; applying the emulsion to graphite electrode surface; and subjecting bacteria in the emulsion to an electric current conducted through the electrode surface.

20. The method of claim 12, wherein the light comprises filtered light of a wavelength range optimized for H₂ production.

21. An expression cassette comprising:
a) a nucleic acid encoding structural proteins of an endo-hydrogenase operon;
b) a heterologous nucleic acid encoding a transcriptional repressor/activator protein responsive to an inducer; and
c) a heterologous nucleic acid comprising bidirectional promoter regions;
wherein the heterologous nucleic acid comprising bidirectional promoter regions is located in operable combination with the nucleic acid encoding the structural proteins on one side and the heterologous nucleic acid encoding the transcriptional repressor/activator protein on another side, and wherein the transcriptional repressor/activator induces expression of the structural proteins in the presence of the inducer.

22. A vector comprising the expression cassette of claim 21, a bacterial origin of replication, and a coding region of a selectable marker in operable combination with a regulatory sequence.

23-27. (canceled)

28. The vector of claim 22, wherein the heterologous nucleic acid encoding the transcriptional repressor/activator protein and the heterologous nucleic acid comprising the bidirectional promoter regions comprise Pseudomonas putida nahR coding region and Pseudomonas putida nahR/nahG promoter regions, and the inducer comprises salicyclic acid.

29. A kit for inducible expression of an endo-hydrogenase in a phototrophic bacterial host cell, comprising the vector of claim 22, and a small molecule inducer.

30. (canceled)


SEQUENCE LISTING (40 sequences)

SEQ ID NO: 1 - DNA, 8152 bp, A. caulinodans
cagcatgaggcggctcagcagatagtccccgcggaataactggacgatgttctggatgacggcgtcccgcatgaccgggaagccggggtcgcctcgcacatgggcggcgaacgcggcgaggtcgactggcgcagcccggagctgaatgcgcccggcgtcaaggccggcagccggcagtcgctgggcgagagcgttcatccatgatccccgatcagcaggcgcgacgcaccagctaccccgacgcaagggccatgccctagccggtgcatcgacgatctcatcttatcatggtgccgttgcgcataaaccctgatcccatcaatgggcattgatgttcgggcgcctgttggtcatgggcgcgtatccagggttttattcacacgcatatcaacgatagcgggtgatgtcgcggagcgtcaagtctgcgctcctcatctttcgggaggtatgatggcgcgatgcgctcctctggtgcaagcgtcttgtcgccggacaggcggagaaaaacgggtcgatccccatggccctgcggacgctgcccctgcgtcctgcgaccgggccggatatcggaatgcatcgcgatcccgacctttcatcgtgcgagccgtggcggcgcacccgtgcggcctgtgcgtcgtcattggatgatgtttaaaacatcataattttatgtttgatattcactcctgacgtgattaggctaattcatcaattctcagccatgggtctttcggccacatgatcaccgctgcgcaactgcgtgccgctcgcgccctgctcgggctcgaccagaaggcgctggccgcgctgtgcgcgctctccgtgccgaccatccagcggatggaggccagtgacggggtcattcgcggcaatgtggattccctgatgaagctcgtgggcgcgctggagggcgcgggcatccagctcctcagcgagggcatggtgagtgaagggggcgggcgcggcgtgcgtctgcgcgaggcccccgtccgccgcccggcgccgcttccggacgaggcctgacccatgcctgtgatcgtcgagctctggtgcatctgtgcgctgctggctctggcggtcgcagccattcccctgtcgcgccatggcggtgcgcatgcggtgatctatggtggcacggggctcatctgcctcgtgggcctgttgaacgctctcacggccctgccggccagccccaccacgctcgcgttgccgctcggcctgccgggcctcggcacgcaggtgcgcctggatgcgctgagtgcggtcttcctggccatcgtcaatctgtgcggcgcggctgccagcctctatgccatcggctatggccgccatgcggcggagcccgcgcgggtgctgcccttctttcccgcctttcttgccgccatgagcctcgtgctgctggcggatgacgcctacagcttcctgttcggctgggaggtgatgtcgctcgcctcctgggcgctggtgctggcggatcatcgggaggccgccaaccggcgggccgccttcatctatctcgtcatggcgagcgccggcgcgttcctgctgatgctcgctttcggacttctggcgggcagcgcggggagcgtggccttttccgtcctgcgcgcccatgcgccggagggtgtgctgggtgccgtcgtgctgtttcttgcgctggcgggtgccggctccaaggcgggcctgtttccgctgcatgcctggctgccgctcgcccatcccgctgctcccagccacgtctcggcgctcatgagcgcgggcatgaccaaggtggcggtctatggcttcgtgcgcatcgcttttgatctactgggcccgcccgaatggtggtggggcatgcccgtgctggccatcggcgccgcgacggcggtgctgggcgtgctctacgcgctgatgcaggcggacctcaagcgggcgctcgccttcagcaccatcgagaacatcggcatcatcttcgtcgggctcggcctcgcgctcgccttctcggccaacggcatgaaagcggcgggggcgctggccctgacggcggcgctgttccacgcgctgaaccacgcgttgttcaagagccttctgttctttggcgccggcgcggtactgcacggcacgggaacacgggacatggagcggctcggtggccttatccatgccatgcctgtgacggccttcgccatgctggtggcctgcgcggccatttcggcgctgccgccgctgaacggcttcgtgtccgagtggctcaccttccaggccatattgctgagcccggacctgcccgcctgggggccgaagctgctggtgccggcggccggcgccctgcttgcccttgccgcggcgctggcggcggcctgcttcgtgcgcatctacggcaccgtcttcctcggccgcccgcgctcgcccgctgccgcgcaggcccatgaggtggagcgctggtcgctggccgccatgctggggctggccgggctgtgtgtgcttgccggcatcctgccgggggtggtgattgatgcggtggctcccgccgtccaggcgctggtggatgcgcgcatgcccccgcaggcgggcaccgcctggctttccatcattcccgtggccgagagccgcagctcctataacgggctgctggtgctcctctttgtgctcggctcggctggcctgatggcgctgctcatccatcgtttcgcgtcgcgcgcggtgcggcgcggcccggcctgggactgcggcttcccagatgcaggcctgcgcacgcagtatagcgcgtcgagcttcgcccagcccatccgccgcgttttcggcaccaccctgttcggtgcgcgcgagcgcatcaccatgccgccgccgggcgatctccggccggcgcgtttcgtgctgcgtatcgtcgatccagcgtgggaggggctctatttgccgctggcgcggggcgtgtggcgcctctcggggcggatggacgccctgcaatatctctccatccggcgctatctcggcctcgtcttcggggcgctggtgctgctgcttctggtgctggcgctatgggcctgatccacgctctcgtcgtgcagggcctccagatggcccttgtgctcgcgctggcgccgctgctcaccggcttcgtgcgcgtgatgaaggcccgcctgctgcgccggcgcgggccgccgctgctgcaaccctatttcgatctcgcgcgcctcgcccgtaaggatgtggtgctggcgggcaatgcctcctggctgttccgagccgcgccctatctcatcttcgcctccacctgggtggcggcggcattggtgcccactttcgccaccggcctcgtcttcagttggacggcggacctcatcgccatcacggctttgctcggcagcgcccgtttctttcaggcgctggcgggcatggacatcggcaccagttttcgcggcctcggctccagccgggaggtgatgatcgcctcgctcgccgagcccgcactcatcatgatcgtctccacgctggcgctggtcgcgggctcgacgcagctctccaccgtcgccgaacacatgctggcggcccatgtgggcctgcgtgtctcgttggggctagcactgattggcctcaccatggtggccattgccgagaatgcgcgcatcccggtggacaatccggcgacccatctcgaactcaccatggtgcatgaggctatggtgctggaatattccggacgccatctcgcgctcatcgagctcgccgcatcgctgaagctggtgctctatctctccctcatcgcctgcgtgttcgtgccgtggggccttgccccggcgggcgcggcgccgctggcgctcctcatcggcatgctggcctatggggcgaagctcgcgctgggcggcagcctgctggccctgtttgagacctccatcgccaagatgcgggtcttccgtgtgccggatttcctcggcgcggcgctgatgctggggctgctcggcacgctgctgctgttcgtgtcgcggagcttctgagcatgggcagccttctcttcgatgtggcgcatcttctggcgggcgggctcgtcttcaccagcctcgtgatgctctatcaggcccgcctgccgaacctcatccgcgtctatggccttcaggcgctgctgctcaccctctccgtggcctggcaggcctatgcacaggatgcgccgcatctctatgtgaccgccgccatcacgctcgccttcaaggcgctcatcattcccgtggcgctgctgcggatgatcgagcggctcggcattcaccggcaggtggaatccgcgctcagcaccggcctcaccatgatggccggcatcggcctcattgccctgtccatggtggtgatgctgcgcgtgacttccggggccgatcctctggcgcgggaggacctcgccttcgccctttccgtcgtgctgctgggcctcctgatgatggtgacgcggcgcaacgccgtcagtcaggtggtgggcttcatgtcgctggagaacgggctgatcctcgccgcgaccggtgccaaggggatgccgctggttgtggaaatctcggtcgccttctcggtgctggtggccttcatcgtcatcggcatcttcctcttccgcatccgcgagcgcttcgacacggtggattccacggcgctcgaccgtttccgggagggccgcagatgaccctcggccttgatgcggcttcggccttgcttctgctgcccgccgccagcgcgctcctgctgttggtgctgccggcgccccgcctcggggcatggctgaatgcggcgagcgccgccttgtccttcgtcgcggcggcctcgctgctgctggtgcagcccccggagggagagttcctgcgggtggacgatctcaacatcgtctttctggtgctgaacagcttcgtgggcgcgaccacgtcgctctacagcgccggctatatcgcccatgagctggagacgggccgcctctcgcccgcgcagatgcgcgtctatcacgccatgtatcaggtgctgctgttcgccatgaatctggcgctgctggcgagcaatctcggcgtgatgtgggtggcgatcgagttggcgacgctcaccaccgtgctgatggtaggcctctatcgcacgcccgccgcgatcgaggcggcgtggaaatatttcatcctcggcagcgtcggcatcgcgctcgccttgttcggcaccatcctcgtctacatggccgcccggccagtggtgggggaggggcacgacggcatgctctggaccgtgctcatgagccatgcggcggccttcgatccggcgctgctcaacctcgccttcgtcttcctgctgctgggctacggcaccaaggtcggcctcgtgccgctgcactcctggctgccggacgcccatgcggaggggccgacgcccatttccgccgtgctctcgggtctcctgctgaacgtagcgctctatgcggtgctgcgcttcaagctgctgctggcggccgcgccgggcgcgctggcgccggggccgctgctcatggggcttgggctgctgtcgctgctgttcgccgggctgatgctctaccggcggcgggacatcaagcgtctgttcgccttctcctccatcgagcacatggggctgatcgcgttcgcgttcggtatcggcgggccggtggcgaatttcgccggcctgctgcacatggtgatgcatagccttaccaagtccgccatcttcttcggcgtcggccatgcggtgcaggtgaagggcacgcaggcgctctccgagatccggggcctcaccgccagccatccgctgctcggctgggggctggtggcgggcgtcgccgccatcgtcggcctgccgccggcaggggtgttcatgagcgaattcctcattctcacctcgaccgttgcgcgggcgcccgtgctggcggggctgctggcgctggggctcgtcatcgccttcgcggccctgatgctgcgcctgcatgcgctggccttcggcgcgccgcgcgggccttcggcaccggtgaagggcacgcaagtgcccatgctggtgcatctggcgctcgtgtggtgcgcgggtgtgtggctgccggggccgctggtaagctggttccagcacatcgcgcatcttctgggctgagggggcgagatgggcgcactcaccgatattctcgccaccggccgcatccgcaccgaccaccggccgtggctgcgcgccgaagtggacgccgcgacatgggaggcgctgtccgaggggctggcggcgggccgctggacgctcatggccctgtggggcgagcccgacgccgtgcacatggcgctgctggatgaggccgcgacctcgctgggcacggccacctatcttgcgggcgacggcacgttcccctccgttggccgccaccatgcgccggcccaaaggctggagcgcaccattcgcgacctgtacggcctggtgccagtcgaagcgcccgatacgcgcctctggctcgaccacggccgctggccggtgtcgcgtcctctcagtggggcccctattccgcccgacacgaacccctatgccttccttcccgtcgatgggcaagggctgcatgaagtggcggtgggccccgtgcatgcgggcatcatcgagcctggccatttccgcttcaccgccaatggcgaggccgtcgtgcggctggaagagcggctcggctatgtccatcgcggcatcgaggggctgatgcgcggggcatccctggagcgcgccgccaccctcgccggccgggtgtctggcgacagcacggtggcctatgccttggccttcgccggcgccgttgaagccgccctcgagatggaagcaccgccacgcgcccgctggctgcggggcctgatggcagagctggaacgcctcgccaatcacttcggtgacatcggtgccgtctgcaacgacgctgcctttgcgcggatgctcgccccctgcgaggtgctgcgcgaacagatgctgcgtgcgagcgaggcggccttcggccagcgcatgatgcgcgacctggtcgtccccggtggcgtgctgatcgatctcgcgccgcgcggagagggccggctgcgttcgctggtggaaactgcgcgttcggccctgccggaactgctggcgctttatgacggcaccgcctccttgcaggaccgcaccgtgggtacgggcatcgcgcagccggatctggtgcgccgcttcggttgcggcggcttcgtggggcgggcatcgggacggggcttcgatgtccggcggacgctaccttacgctccttacgatgcgctgcgcttcgacgtggcggtgctggaggcgggagatgtggacgcgcgggtgcgcatccgcttcagcgaggcggaagagagcctttcgctcattacgcagatgctcagcaatatgcccgacgggggcgacctgtcttcgccgctgccgcagtccggcgcggcgcgcgagggcgtggcactggtggaagggttccggggcgacgtcttcgccttcgtgcggcttgatgcggccgggcgcgtcgcccatgcccatctgcgcgatccctcctggttccagtggccggttctggaggcggtcatcgaaggcaatatcatcgccgatttccccctctgcaacaaatccttcaactgctcctatgcggggcacgacctgtgaggcggcgcggatgcggacactcctgatcgatgccctgctgaaaggcccgctgaccgagccggcgccggcggcggatgatgcggagctgctggccttcggcgcgcagctcgaaggggcggcgcggcgcaggctcggccgcggtcttgccatccggcaggtggatgcgggctcctgcaacggctgcgaactcgaaatccatgccctctccaacgcctattacgatctggagcgcttcggtctgcgtttcgtcgcatcgccccgccatgcggacgtcctgctggttacggggccggtgacgaccaacatgcgggaggcgctggtgcggacctatgaggcgacgcccggcccgaaatgggtggtcgcggccggaagttgcgcctgtgacggtggcctgttcgcgcagagctatgcctgcgccggggcggtggagaatgtcattccggtcgatctgcgtatccccggctgcccgcctgccccgctcagcctgctcaagggcctgatcgcccttctgcaaagggattagccaacacgtcccggtgcattt

SEQ ID NO: 2 - protein, 94 aa, A. caulinodans
MITAAQLRAARALLGLDQKALAALCALSVPTIQRMEASDGVIRGNVDSLMKLVGALEGAGIQLLSEGMVSEGGGRGVRLREAPVRRPAPLPDEA

SEQ ID NO: 3 - protein, 667 aa, A. caulinodans
MPVIVELWCICALLALAVAAIPLSRHGGAHAVIYGGTGLICLVGLLNALTALPASPTTLALPLGLPGLGTQVRLDALSAVFLAIVNLCGAAASLYAIGYGRHAAEPARVLPFFPAFLAAMSLVLLADDAYSFLFGWEVMSLASWALVLADHREAANRRAAFIYLVMASAGAFLLMLAFGLLAGSAGSVAFSVLRAHAPEGVLGAVVLFLALAGAGSKAGLFPLHAWLPLAHPAAPSHVSALMSAGMTKVAVYGFVRIAFDLLGPPEWWWGMPVLAIGAATAVLGVLYALMQADLKRALAFSTIENIGIIFVGLGLALAFSANGMKAAGALALTAALFHALNHALFKSLLFFGAGAVLHGTGTRDMERLGGLIHAMPVTAFAMLVACAAISALPPLNGFVSEWLTFQAILLSPDLPAWGPKLLVPAAGALLALAAALAAACFVRIYGTVFLGRPRSPAAAQAHEVERWSLAAMLGLAGLCVLAGILPGVVIDAVAPAVQALVDARMPPQAGTAWLSIIPVAESRSSYNGLLVLLFVLGSAGLMALLIHRFASRAVRRGPAWDCGFPDAGLRTQYSASSFAQPIRRVFGTTLFGARERITMPPPGDLRPARFVLRIVDPAWEGLYLPLARGVWRLSGRMDALQYLSIRRYLGLVFGALVLLLLVLALWA

SEQ ID NO: 4 - protein, 318 aa, A. caulinodans
MGLIHALVVQGLQMALVLALAPLLTGFVRVMKARLLRRRGPPLLQPYFDLARLARKDVVLAGNASWLFRAAPYLIFASTWVAAALVPTFATGLVFSWTADLIAITALLGSARFFQALAGMDIGTSFRGLGSSREVMIASLAEPALIMIVSTLALVAGSTQLSTVAEHMLAAHVGLRVSLGLALIGLTMVAIAENARIPVDNPATHLELTMVHEAMVLEYSGRHLALIELAASLKLVLYLSLIACVFVPWGLAPAGAAPLALLIGMLAYGAKLALGGSLLALFETSIAKMRVFRVPDFLGAALMLGLLGTLLLFVSRSF

SEQ ID NO: 5 - protein, 218 aa, A. caulinodans
MGSLLFDVAHLLAGGLVFTSLVMLYQARLPNLIRVYGLQALLLTLSVAWQAYAQDAPHLYVTAAITLAFKALIIPVALLRMIERLGIHRQVESALSTGLTMMAGIGLIALSMVVMLRVTSGADPLAREDLAFALSVVLLGLLMMVTRRNAVSQVVGFMSLENGLILAATGAKGMPLVVEISVAFSVLVAFIVIGIFLFRIRERFDTVDSTALDRFREG

SEQ ID NO: 6 - protein, 481 aa, A. caulinodans
MTLGLDAASALLLLPAASALLLLVLPAPRLGAWLNAASAALSFVAAASLLLVQPPEGEFLRVDDLNIVFLVLNSFVGATTSLYSAGYIAHELETGRLSPAQMRVYHAMYQVLLFAMNLALLASNLGVMWVAIELATLTTVLMVGLYRTPAAIEAAWKYFILGSVGIALALFGTILVYMAARPVVGEGHDGMLWTVLMSHAAAFDPALLNLAFVFLLLGYGTKVGLVPLHSWLPDAHAEGPTPISAVLSGLLLNVALYAVLRFKLLLAAAPGALAPGPLLMGLGLLSLLFAGLMLYRRRDIKRLFAFSSIEHMGLIAFAFGIGGPVANFAGLLHMVMHSLTKSAIFFGVGHAVQVKGTQALSEIRGLTASHPLLGWGLVAGVAAIVGLPPAGVFMSEFLILTSTVARAPVLAGLLALGLVIAFAALMLRLHALAFGAPRGPSAPVKGTQVPMLVHLALVWCAGVWLPGPLVSWFQHIAHLLG

SEQ ID NO: 7 - protein, 504 aa, A. caulinodans
MGALTDILATGRIRTDHRPWLRAEVDAATWEALSEGLAAGRWTLMALWGEPDAVHMALLDEAATSLGTATYLAGDGTFPSVGRHHAPAQRLERTIRDLYGLVPVEAPDTRLWLDHGRWPVSRPLSGAPIPPDTNPYAFLPVDGQGLHEVAVGPVHAGIIEPGHFRFTANGEAVVRLEERLGYVHRGIEGLMRGASLERAATLAGRVSGDSTVAYALAFAGAVEAALEMEAPPRARWLRGLMAELERLANHFGDIGAVCNDAAFARMLAPCEVLREQMLRASEAAFGQRMMRDLVVPGGVLIDLAPRGEGRLRSLVETARSALPELLALYDGTASLQDRTVGTGIAQPDLVRRFGCGGFVGRASGRGFDVRRTLPYAPYDALRFDVAVLEAGDVDARVRIRFSEAEESLSLITQMLSNMPDGGDLSSPLPQSGAAREGVALVEGFRGDVFAFVRLDAAGRVAHAHLRDPSWFQWPVLEAVIEGNIIADFPLCNKSFNCSYAGHDL

SEQ ID NO: 8 - protein, 170 aa, A. caulinodans
MRTLLIDALLKGPLTEPAPAADDAELLAFGAQLEGAARRRLGRGLAIRQVDAGSCNGCELEIHALSNAYYDLERFGLRFVASPRHADVLLVTGPVTTNMREALVRTYEATPGPKWVVAAGSCACDGGLFAQSYACAGAVENVIPVDLRIPGCPPAPLSLLKGLIALLQRD

SEQ ID NO: 9 - DNA, 1326 bp, P. putida
ggaacggtgtcctgtctttcatggggctcgttcactgctaacttacctgcttccctgtctgtcaattctctctatcctgcgatcccgcgaagaaccaaaaaagctcgacagagggcgcggtcattttaggtcgggcggatcggcgccgccggctcggctggtgtgccgcacagcaccgcctacgtgagctgccagttgatgaacttcccccgttgccagctagggcgcaagcgggctgtataagatcactgcccatcacattgatcggctcggattttttctcaatccgtaaacaggtcaaacatcggttgcctgcaaccaaatattggctaggtccttgtggtaccttcgcatgccagaacatgttgatggctatttcaggcaagacgactgggtgcggcaaggcgcttaggccgaagggctccacgcagcagtcggctaaacgtatcggcacagtggcgagcagatcggtgcgctggaggatgtggccaacggcggcgaagtgcggcacttccagacggatgtcgcgccggatgccgacccgtgtcatgtacgtgtccacctcgccgtggccggtgccagcggcgatgacacgcacgtggccgtaggaacagaagcgctccagagtcaggggttcgcgggtgactggatggtccttgcgacataggcacacgtagtgattctggagcagccggcgctgaaagaagccagtttgcagattgggaagcaggcccacggccaagtccacggttccgttctgcaaggcctgcatcaggctcatcgaactgtcgcgcaccgtactgatcacgcaattgggggcctggtgagccagcacatccatcagccgcggcatgaagtagatctcgccaatgtcggtcatggccagggtgaaggtacgctcgctggtcagcggatcgaagctttcatggtgctgtagggcgttgcgcagtgcgtgcatggccgaagtgacgggctcggccagatgcgcggcatagggtgtgggttccattccctgatgtgtgcgcacgaagagtgggtcctgtagcgaggtgcgcaggcgtttcagcgcattgctcacggcaggctgggtcaggcccaggttctccgcagtgatagagacgcgtctgtcgaccagcaactggttgaacaccaccagcaggtttaaatccaggtcacgcagttccatggggcctcgcttgggttattgctggtgcccggccgggcgcaatattcatgttgatgatttattatatatcgagtggtgtatttatcaatattgtttgctccgttatcgttattaacaagtcatcaataaagccatcacga

SEQ ID NO: 10 - protein, 672 aa, R. palustris
MTELAVQLWCVAVLLAIAVIAVPLSRRPGLATATIYSATLVTCAVATASALTWLLLHADAAAQRTLPIGLPWLGAHLRLDALAAFFLVVVNLGGTLASLYALGYGRHEHAPHRVLPFYPAFLAGMNMVVLAADAFSYLLSWEFMSLASWALVMAHHRDGSNRRAGYVYLLMASFGTLALLLAFGLLAGAAGNYDFAAIRAAQHAPWIATLVLILLLLGAGSKAGLVPLHVWLPLAHPAAPSHVSALMSGVMTKVAVYGFIRVVFDLLGTPDWSTSGIVLALGGITAALGILYALMEKDLKRLLAYSTIENVGIIFVSLGLALAFQANHQPLPAALAFTAALFHVLNHSLFKSLLFFGAGAVLSATGERDMDKLGGLIHRMPVTSVVFLIGSVAISALPPFNGFVSEWLIFQAVLQSPQLPQWGLKILVPAVGGLMALAAALAAACFVKAYGVTFLGRPRGAATLAAKEVDRFSLAAMAILAALCLAAGVLPGLVMDTLAPIATEILGSRLAPQTPLAWLSIVPIAQSHSSYNGLLVALFIAVSASLLFVAIRLFSSGAVRRGPAWGCGFTEPAPLAQYSAAGFAQPIRRVFGTLIFRARDHVTMPPPGDTAPARLRIELHDQIWERIYAPLAGGVMAASVRLNRMQLLTIRGYLSLVFATLVTLLLVLAIWT

SEQ ID NO: 11 - protein, 316 aa, R. palustris
MITDIAVQGVQMLLVFLLAPLLTGFIRKVKARLQRRRGPSLLQPYRDLIRLLRKEVVLADNASWLFRATPYVTFAAIWVAAALVPTFATGLLFNWTADLIVIVALLGTARFFLALAGMDVGTSFGGIGSSREVMIAALAEPAMLMIVFCVALVAGTTHLSTVANYIASTAVGLRVSLGLAMIALVMVALAENARIPIDNPSTHLELTMVHEAMILEYSGRHLAMIEYGAQLKLLLYVSLIVCLFYPWGIGVFGAGPAAYLAGAALYLFKITIAGFLLAVFETATAKMRVFRVPQFLGAALMLGLLGTLLLFVSKGL

SEQ ID NO: 12 - protein, 220 aa, R. palustris
MHGLAFDISHSLAGGLVLVSFMMLYQDRLTSLLNVFALHAIMLSLAVGWQALIQDAPHLYVTALIALLFKAIIIPVALRRIVYKLGIHRDIETVVGIGPTMLAGMGLVALSLALMLRVTADADALAREDLAFALSVVLLGLLFMVTRRNAVSQVIGFMSLENGLILAAAGAKGMPLVVEISVAFSVLIAFIVIGIVLFRIRERFDSVDVAQLDVSRGEQR

SEQ ID NO: 13 - protein, 483 aa, R. palustris
MNLSGLDPVVGVLLIPVCAAIVLALLPGYRLTARLNVVASLGSFLCAASLLVLPRPLPGPYILVDDLNIVFIVLNTFVGFTTSLFSASYIAHELETGKLTPTYLRFYHAMYQIMMFGMNLAFMSNNIGLMWVAVELATLTTVLMVGIYRSPAALEAAWKYFILGSVGIALALFGTILVYMAARPVVGEGQDGMIWTVLVARAAGFDAPLLNVAFVFLFLGYGTKVGLAPLHAWLPDAHAEGPTPISAVLSGLLLNVALYALLRFKMLLAANGDAMPPGVLMIVMGLASLLFAAFMLYRRRDIKRLFAYSSIEHMGIIVFAFGMGGPLANFAGLLHMVMHSLTKSAIFFAVGHITQVKGTQSLAKIRGITETHPVLGWGLVVGVAAIAGLPPLGIFMSEFLLVSSTFARAPWLALLLVFGILVAFGALMLRLTGIAFGRPRGSRQPVQASYVPMFAHLALVLIAGVYLPAPVVAWFQHVAKLLG

SEQ ID NO: 14 - protein, 503 aa, R. palustris
MPSLIDLNLEGRQVARHRPWSRTVVDSAVWSFAVKMLSERQWQLLSLWGEPAAVHMALFDPRTAEIGLISLDCPHGSFPSVAAKHLPALRLERTITDLFGLKPDGAIDDRPWLDHGQWGVHAPLGARVQAETPPPAYRFLPVHGEGAHQVAVGPVHAGIIEPGHFRFTVSGETVVRLEQRLGYVHKGIDGLIAGADLASAARLAGRVSGDSTVAYALAFSRAVEAAAEIVVPARAVWLRALVAELERLANHLGDIGAVCNDAAFAIMLAQFGVLREHVLRAADAAFGHRLMRDVVMPGGVARDLDDNGKAAFRDALAAVRAKLPALIELYDSTASLQDRTVSTGILRPALADQYAAGGFVGRASGRGFDARRTLAYAPYHQLRFDVPVLTEGDVDARIWIRIREVEQSLSLVDQILDRLPRGEIRVAPELPDQACEGLALIEGFRGDILVWLRLQDGVVARCHLRDPSWFQWPLLEAVIEGNIIADFPLCNKSFNCSYSGVDL

SEQ ID NO: 15 - protein, 177 aa, R. palustris
MRKLLFESLFAKPLTESPPAPDDAAVAELAARLDATARRMLGRSLSIREVDAGSCNGCELEIHALNNAYYDVERFGLRFVASPRHADVLMVTGPVTHNMREALRLTYDAVPGPKWVVAVGDCAKDGGCFAGSYAVAGGVSAVIPVDLHIPGCPPSPMQILRGLLALLDSVSERETSR

SEQ ID NO: 16 - protein, 672 aa, R. palustris
MISAAGAQLYCVAALLAVALLAVTLAGKRAATVVVYVATLLICVAGCGGALGFLLGGIAKPDLVTLPIGLPWLGAHFRLDALSAFFLAVINLGGALASLYAIGYGRHEHAPQRVLPFFPAFLAGMNMVLLADDAFSYLLSWEFMSLASWALVMAHHRQSGNARAGYVYLVMASFGTLALLLAFGFLAGPAGDYDFAAIRAAPHSSAAATLVLILMLLGAGSKAGIVPLHVWLPLAHPAAPSHVSALMSAVMTKVAIYGFIRVVFDLLGPPEWTTALVVLTLGGVTAVMGILYAMMEKDLKRLLAYSTIENVGIVFVSLGLALAFQANGQAVPAALAFTAALFHILNHALFKSLLFFGAGSVLVATGERDMDKLGGLIHRMPVTAVVFLIGSIAISALPPFNGFVSEWLIFQAVLQSPQLPQWGLKIMVPAIGGMLALAAALAAACFVKAYGTTFLSRPRSKAAEAAHEVDRFSLTAMAILAALCLLAGIFPGPVMDALAPVTSQIVHSRLGGQSELPWLSIIPLDARKGSYNGLLLLGFITVSATLGWLAIHRLASHATRRSPAWGCGFNDAAPQAQYSPDSFAQPIRRVFGTLLFRARDHVEMPPPGSVAPARFSIEIHDLIWENLYGRIAGAVNAAATKLDAMQLLTIRGYLSLVFATLVTLLLVLAIWS

SEQ ID NO: 17 - protein, 319 aa, R. palustris
MVVILDDILVQGFQMLLVFMLAPLLTGFVRKVKARLTRRQGPSLLQPYRDLLRLLRKEAVLADNASWLFRTTPYLTFAAIWVAAALVPTFATGLLFNWTADLIVIVALLGTARFFLALAGMDIGTAFGGIGSSREVMIAALAEPAMLLIVFGVALVAGSTQLSTVANFIASSQVGLRVSLGLAMIALVMVALAENARIPVDNPSTHLELTMVHEAMILEYSGRHLAMIEFGAFLKLLLYVSLIGCLFFPWGIAIAGDAPLSYVVGAGLYLLKVTVAGFLLALFETATAKMRVFRVPQFLGAALMLGLLGTLLLFVSRSL

SEQ ID NO: 18 - protein, 220 aa, R. palustris
MHGLAFDISHSLAGGLVLISFMMLYQDRLYSLLNVFALHAVVLSLAVTWQAFIQDAPHLYVTAAIALVFKAIIIPVALHRIIRQLGIHRDIETVVGIGPTMLAGMGLVALSMVIMLRVTADADALAREDLAFALSVVLLGLLVMVTRRNAVSQVVGFMSLENGLVLAATGAKGMPLVVEISVAFSILIAFIVIGIFLFRIRERFDSVDVGALDDSRGERR

SEQ ID NO: 19 - protein, 483 aa, R. palustris
MNFAAFNPVLAVVLIPIFAAALLAALPGYRLTARLNVVACLATFVSAVALLILPRPAPGPYVLVDDLNIVFIVLNSFVGLTTSLFSASYIAHELETGRLTPAYLRFYHAMYQTMMFGMNLAFMSNNIGLMWVAVEVATLTTVLMVGIYRTEAALEAAWKYFILGSVGIAFALFGTILVYMAATPVIGEGQDAMIWTLLIERASKFDPALLNVAFVFLFLGYGTKVGLAPLHAWLPDAHAEGPTPISAVLSGLLLNVALYALLRFKMLMAANPEAMAPGTLMVTMGLASLLFAAFMLYKRRDIKRLFAYSSIEHMGIIVFAFGMGGPLANFAGLLHMVMHSLTKSAIFFAVGHVAQIKGTQNIADIRGLTESHPALGWGLVAGVAAIAGLPPLGIFMSEFLVVSSTFARQPLLALLLVFGLLIAFGALTLRLTGIAFGEPRGSTQPAQASHVPMFAHLALVLVAGVYLPAPIVIWFQHVAKLLG

SEQ ID NO: 20 - protein, 502 aa, R. palustris
MSSLSDWKAEGHPVAQHRPWPRVVVDAVEWSRAILELASGRWSLLALWGEKAVVHMAMLEAHLGKVVVLSLNCPDGSFPSVGMLHPPALRLERTITDLFGLVAENAPDPRPWLDRGQWGLRYPLGDRAPAAPPRAYEFLPVEGEGVNQIAVGPVHAGIIEPGHFRFTANGETVVRLEQRLGYVHKGIEGLMAGADLVRGAELAGKVSGDSTVAYGYAFARAAEAALEIDAPPRAVWLRALCAELERLANHLGDIGGICNDAAFALMLAHCSVLRETVLRAADKAFGHRLMRGVIVPGGVARDLDDTGKGTIRQAVKTIRQRFPELVELYDETASLQDRTVATGNLSAVLAEQYAVGGYVGRGSGRAFDTRRKLAYPPYDRLRFEVPVLTEGDVNARVWIRIREVEQSLSLIDQLLADLPKSDWLAPLPPRAEAVEGMAVVEGFRGDVLVWLRLAAGRIERCHLRDPSWFQWPLLEAAIEGNIVADFPICNKSFNCSYSGVDL

SEQ ID NO: 21 - protein, 173 aa, R. palustris
MRKLLFESLFRKPLTEAAPSLDEAAVAELASALGDVSRRKLGRSLAIREVDAGSCNGCELEIHALNNAYYDIERFGLRFVASPRHADVLLVTGPVTKNMADALKTTYDATPSPKWVVACGDCARDGGCFAGSYAVVGGVSQVIPVDLHIPGCPPPPLQILRGLLALLDNAAAR

SEQ ID NO: 22 - protein, 672 aa, R. palustris
MTELAVQLWCVAALLAAAVVAVPLSRWPGLSTAIIYSATLMACAIATASALTWLLLHADAAAERTLPIGLPWLGAHLRLDALAAFFLVVVNLGGALASLYALGYGRHERAPHRVLPFYPAFLAGMNLVVLAADAFSYLLSWEFMSLASWALVMAHHRDGGNRRAGYVYLVMASFGTLALLLAFGLLAGTAGDYDFASIRIAPHSPYVATLVLILMLLGAGSKAGLVPLHVWLPLAHPAAPSHVSALMSGVMTKVAIYGFIRVVFDLLGQPDWSTSGIVLALGGITAALGILYALMEKDLKRLLAYSTIENVGIVFVSLGLALAFQANDDKVPAALAFTAALFHIFNHSLFKSLLFFGAGAVLSATGERDMDRLGGLIHRMPFTSVVFLVGCVAISALPPFNGFVSEWLIFQAVLQSPQLPQWGLKILVPAVGGLMALAAALAAACFVKAYGVTFLGRPRGAATIAAKEVDRFSLAAMAILAALCLLAGVLPGVVMDALAPIATNILGSRLAPQSGMAWLSIVPISGNHSSYNGLLVAMFIALSALLLFVAIRMFSSGAVRRGPAWGCGFTDPAPLAQYSADGFAQPIRRVFGTLIFRARDHVTMPPPGDTAPARLTIELHDQIWERIYAPLAGGIMIASARLDRLQLLTIRGYLSLVFVTLVTLLLVLAIWT

SEQ ID NO: 23 - protein, 316 aa, R. palustris
MIADIAVQGLQMLLVFLLAPLLTGFIRKVKARLQRRRGASMLQPYRDLIRLLRKDVVLADNASWLFRATPYVTFAAIWVAAALVPTFATGLMFNWTADLIVVVALLGTARFFLALAGMDIGTSFGGIGSSREVMIAALAEPAMLMIVFCVALVAGSTHLSTVANYIASTEVGLRVSLGLAMIALVMVALAENARIPVDNPSTHLELTMVHEAMILEYSGRHLAMIEFGAQLKLLLYVSLIVCLFYPWGIGIFGAGPLAYLAGAALYMGKLTVAGFLLAVFETATAKMRVFRVPQFLGAALMLGLLGTLLLFVSKGL

SEQ ID NO: 24 - protein, 220 aa, R. palustris
MHGLAFDISHSLAGGLVLVSFMMLYQDRLTSLLNVFALHAVMLSLAVGWQALIQDAPHLYVTALIALLFKAIIIPVALRRIVYKLGIHRDIETVVGIGPTMLAGMGLVALSLALMLRVTADADALAREDLAFALSVVLLGLLFMVTRRNAVSQVIGFMSLENGLILAAAGAKGMPLVVEISVAFSVLIAFIVIGIVLFRIRERFDSVDVSQLDVSRGEQR

SEQ ID NO: 25 - protein, 483 aa, R. palustris
MNLSGLDPVVGVLLIPVCAAAVLAVLPGYKLTAQLNVLASLGSLVCAASLLVLPRPAPGPYILVDDLNIVFIVLNTFVGFTTSLFSASYIAHELETGKLTPTYLRFYHAMYQIMMFGMNLAFVSNNIGLMWVAVELATLTTVLMVGIYRTPAALEAAWKYFILGSVGIALALFGTILVYMAARPVVGEGQDGMIWTVLVSRAAGFDAPLLNVAFVFLFLGYGTKVGLAPLHAWLPDAHAEGPTPISAVLSGLLLNVALYALLRFKVLLAANAETMPPGILMIVMGLASLLFAAFMLYRRRDIKRLFAYSSIEHMGIIVFAFGMGGPLANFAGLLHMVMHSLTKSAIFYAVGHITQVKGTQSLSKIRGITESHPVLGWGLVVGVVAIAGLPPLGIFMSEFLLVSSTFARSPLLALLLVFGLLVAFGALMLRLTGIAFGEPRGSREPVQASYVPMFAHLALVLIAGVYLPAPVVAWFQHVAKLLG

SEQ ID NO: 26 - protein, 503 aa, R. palustris
MPSLIDLNLEGLQVARHRPWSRTVVDSAVWSFAAKMLSEQQWQLLSLWGEPAIVHMALFDPRTAEIGVVSLDCPHRSFPSVAAKHLPALRLERTIRDLYGLVPEGAIDDRPWLDHGQWGVHAPLGEPVEFLAPSPSYRFLPVEGEGAHQVAVGPVHAGIIEPGHFRFTVSGETVVRLEQRLGYVHKGIDGLLAGAELSRAARLAGRVSGDSTVAYALAFARAVEAAAEIAAPARAVWLRALVAELERLANHLGDIGAICNDAAFALMLAQFGVLRENVLRAADAAFGHRLMRDVVTPGGVTRDLDATGTAAIRKALAAVHNKLPELIELYDRTASLQDRTVGTGILKASLADQYAAGGFVGRASGRAFDARRTLGYAPYDELRFEIPVRTEGDVDARIWIRISEVEQSLALVDQILDRLPGGDICVAPALPEQVCEGLALVEGFRGDILVWLRLRDGVVERCHLRDPSWFQWPLLEAVIEGNIIADFPLCNKSFNCSYSGVDL

SEQ ID NO: 27 - protein, 177 aa, R. palustris
MRKLLFESLFAKPLTETAPAPDETGVAELAALVNETARRRLGRSLAIREVDAGSCNGCELEIHALSNAYYDVERFGLRFVASPRHADVLMVTGPVTHNMREALRLTYEAVPGPKWVVATGDCAKDGGCFAGSYAITGGVSAVIPVDLHIPGCPPSPVQILRGLLALLDSVSAREISR

SEQ ID NO: 28 - protein, 689 aa, R. palustris
BMLPVQRSQVPRREDAPVIAAVAVQLVCVAALLAIAVLALPLSRTRAATABVVYGATTLVCAGALIGALRWLFAGAAADELVLPIGLPWLGAHFRLDALAAFFLGVVNLGGAVASLYALGYGQHETAPQRVLPFFPAFLAGMNLVVLAADAFSYLLCWEFMSLASWALVMTHHREPGNARAGYVYLLMASFGTLALLLAFGLLAGPAGDYGFAAIRAMPHAPAEATLVLILMLLGAGSKAGLVPLHVWLPLAHPAAPSHVSALMSGVMTKVAVYGFIRVVFDLLGPPNWTASVLVLSLGGVTAVMGILYALLEKDLKRLLAYSTIENIGIIFVCLGLALAFQANGQTLAASLAFTAALFHVFNHSLFKSLLFFGAGAVLVATGARDMDRLGGLIHRMPVTSVVFLIGCVAISALPPFNGFVSEWLMFQAVLLSPSLPQWGLKIMVPAIGALLALAAALAAACFVKAYGVSFLSRPRSDAAANAQEVDRFSLAAMVILASLCLLAGIFPGLVIDALSPVTQQILDSRLAVQSHLPWLSIIPIAERRGSYNGLLVLAFIAISASLGYLAIRRFSSHAVRRAPAWGCGFSDAVPQAQYSPDSFAQPLRRVFGTLLFAAREHVEMPPPGAVTPARFSVEIHDLVWEGLYGRIAGAVDYAAIRLDRWQLLTIRGYLSLVFVTLVTLLLVLAIWS

SEQ ID NO: 29 - protein, 316 aa, R. palustris
MISDILVQGIQMLLVFLLAPLLTGFIRKVKARLMRRKGASIFQPYRDLLRLLRKEVVLADNASWLFRATPYLTFAAIWVAAALVPTFATGLLFNWTADLIVIVALLGTARFFLALAGMDVGTAFGGLGSSREVMIAALAEPAMLLIVFCVALVAGSTQLSTVANFIASSQVGLRVSLGMAMIALVIVALAENARIPVDNPSTHLELTMVHEAMILEYSGRHLALIEFGAFLKLLLYISLIGCLFFPWEIAVFGAGPLSYVIGAGMYLLKVTVAGFLLAVFETATAKMRVFRVPQFLGAALMLGLLGTLLLFVSKSL

SEQ ID NO: 30 - protein, 220 aa, R. palustris
MHSLSFDISHSLAGGLVLISFMMLYQDRLSSLLNVFALHAVTLSLAVAWQALIQDAPHLYVTAAIALVFKAIIIPVALHRIIRQLGIHRDIETAVGIGPTMLAGMGLVALSMVLMLRVTAEADALAREDLAFALSVVLLGLLVMVTRRNAVSQVVGFMSLENGLVLAATGAKGMPLVVEISVAFSILIAFIVIGIFLFRIRERFDSVDVAALDESRGERR

SEQ ID NO: 31 - protein, 483 aa, R. palustris
MTLPGFNPVLGVVLIPIFAAALLAALPGYRLTARLNVVACLATFVCAVSLLILPRPTPGPYVLVDDLNIVFIVLNSFVGLTTSLFSASYIAHELETGRLTPAYLRFYHAMYQTMMFGMNLAFMSNNIGLMWVAVEVATLTTVLMVGIYRTEAALEAAWKYFILGSVGIAFALFGTILVYMAARPIIGEGQDAMIWTLLIERASQFDPALLNVAFVFLFLGYGTKVGLAPLHAWLPDAHAEGPTPISAVLSGLLLNVALYALLRFKMLMTANPEAMAPGTLMVTMGLTSLIFAAFMLYRRRDIKRLFAYSSIEHMGIIVFAFGMGGPLANFAGLLHMVMHSLTKSAIFFAVGHIAQIKGTQTIDEIRGITESHPALGWGFVAGVVAIAGLPPLGIFMSEFLVVSSTFARQPLLALLLVFGLLVAFGALTLRLTGVAFGEPRGSTQPAEASYLPMFAHLVLVLVAGVYLPAPIVVWFQHVAHLLG

SEQ ID NO: 32 - protein, 502 aa, R. palustris
MPSLSDLKREGRIARQQRPWPRIVVDEVLWSGAATSVAERRLTLLALWGEPSAVHMAVLEPSTAEVAVISLDCPNRCFPSVAAPHPPALRLERSIRDLFGLEPVGSPDSRPWLDHGQWGVTAPLGASTASAPQPAYEFLPVEGNGLHQVAVGPVHAGIIEPGHFRFTASGETVVRLEQRLGYVHKGIEGLMGGASLQQGAKLAGRVSGDSTVAYSFAYSRAAEAALELKVPERAVWLRALMAELERLANHLGDIGAICNDAAFALMLAHCGVLRENVLRAAAIAFGHRLMRDLIVPGGVRGDLSVDGLKAIEAALVSIRHQFPALIELYDDTASLQDRTVGTGVVSSELVEQYGAGGFVGRGSGRAFDTRRKLCYPPYGQLRFDVPVLPEGDVNARIWVRIREVEQSMAMIDQILDKLPGGELLTSVPAIAEDREGLGLVEGFRGDVLVWLRLRDGVITRCHLRDPSWFQWPLLEAAIEGNIVADFPLCNKSFNCSYSGPDL

SEQ ID NO: 33 - protein, 174 aa, R. palustris
MRDQLFQGLFRRPHTEPAPALDPAAVEELATSLGDLARRRLGRSLSIREVDAGSCNGCELEIHALSNAYYDVERFGIRFVASPRHADVLLVTGPVTKNMRDALKATYEATPSPKWVVALGDCAKDGGCFAASYAVEGGVSAVIPVDLHIPGCPPPPLLILQGLLALLARADAGA

SEQ ID NO: 34 - protein, 641 aa, A. caulinodans
MYQAIVFLPLIGFLIAGLFGRVLGPRPSELITTALLFIACLFAWITFIKVGFAGFDTRVQVMHWIYVGDLKIDWALRIDTMTAIMLIVVTSVSSLVHLYSIGYMHEDPSRPRFFAYLSLFTFAMLMLVTADNLVQLFFGWEGVGLASYLLIGFWFEKPSANAAAMKAFVVNRVGDFGFMLGIFAVFVMTGSVSFDTIFAQAPSLAGKTITFLGHHWDAPTVIALLLFIGAMGKSAQFLLHTWLPDAMEGPTPVSALIHAATMVTAGVFMVARMSPIFELSPSALDVVTIVGATTAMFAATVALVQNDIKKVIAYSTCSQLGYMFVAMGAGAYSVGVFHLFTHAFFKALLFLGAGSVIHAMHHEQDMRHMGGLYKKIPFTYGAMMIGTLAITGFPFLAGYYSKDAIIEAAYASHSHFRVYAYWMTVLAAALTSFYSWRLVFLTFHGHPHDHHHYDHAHESPLVMTIPLAVLSVGAVIAGFVCYNLFVGHDVEHFFRSSIFMGPDNHILHAMHEVPGWAAYMPTVMMVLGFVVAYWFYMVDRRVPAALAKSQDALYQFLLNKWYIDELYNFLFVRPALCLGRILWKKGDGAIIDGLGPNGISARVLDVTGQLVRLQTGYLYHYAFVMLVGVAAIITWFMFAGV

SEQ ID NO: 35 - protein, 339 aa, A. caulinodans
MSWQDTLIHVLIILAQSLGLLVGLLIVIAYLLLADRKIWAAVQLRRGPNVVGPLGLFQSFADLIKFVLKEPVIPSGANKVAFLLAPLITCVLALAAWVVVPLADGWVVADLNLGTLYIFAISSLGVYGIIMGGWASNSKYPFLAALRSAAQMVSYEVSIGFVIVTVLMCAGSLDLSKVVHAQDSRLGIFGWYWLPLLPMFVIFFISALAETNRPPFDLVEAESELVAGFMTEYGSTPYLLFMLGEYVAIVTMCALASVLFLGGWLSPIPFAPFTWVPGIVWFLLKVCFMFFMFAMAKAMVPRYRYDQLMRLGWKVFLPISLGMVVLVASFLHFTGLAPQ

SEQ ID NO: 36 - protein, 202 aa, A. caulinodans
MSFAAAFFYLFACILVGSAFMVISSRNPVYSVLFLILAFVNAAGLFILLGVEFLALILVVVYVGAVAVLFLFVVMMLDVDFVELRQGFLQYLPVGAIVGLIFLAELVLVVGTWAAGPGVTVATNAVMTYPRGNTHALGQVLYTDYIYFFQIAGFVLLVAMIGAIVLTLRHKPNVKRQNISAQVARTPETAMEVVKVKPGQGI

SEQ ID NO: 37 - protein, 504 aa, A. caulinodans
MSNWPILSVVTFLPLVGALFIFVVRGEDEVAARNARWVALWTTLVTFVVSLFLLPGFDPAAPGFQFVEKKPWLGGIAAYHMGVDGISLSLVILSTFLMPFCILASWDSVKKRVKEYMICFLVLETLMVGTFCALDLVLFYFFFEAALIPMFLIIGVWGGARRIYASFKFFLYTLLGSVLMMLAIMAMYWKAGTTDVPTLMQYGFPAGMQTWLWLAFFASFAVKMPMWPVHTWLPDAHVEAPTAGSVILAGVLLKMGGYGFIRFSLPMFPDASHLFAPLVYTLSVVAIIYTSLVAMVQEDIKKLIAYSSIAHMGFVTMGLFSMNEAGLQGAMFLMISHGFVSGALFLCVGVIYDRMHTREIAAYGGLVKRMPLYAVAMMVFTMANVGLPGTSGFVGEFLTMLSAFQRNTWVAFFAATGVILSAAYALWLYWRVIFGPLEKENLMKLKDLNAREIGIMIPLIALTIIFGFWPAPVLDMTSHAVNFVVARTETAASVAKTAALLLGH

SEQ ID NO: 38 - protein, 211 aa, A. caulinodans
MDETLKDLAAHVTGALPGAVVAHKIAYGELTLETTPDHILKLATFLRDDPSCLFTCIVDVCGADYPAREQRFEVVYHLLSLKQNARVRVKLSAGEETLVPSVTGIWPGANWFEREAYDLYGILFTGHPELRRLLTDYGFDGHPLRKDFPLTGFVEVRYDDELKRVVYEPVRLAQEFRNFDFLSPWEGVEYILPGDEKASSQPGAPAAPKAS

SEQ ID NO: 39 - protein, 402 aa, A. caulinodans
MAHTLDSQQPAVPVRNFQINFGPQHPAAHGVLRMVLELNGEVVERVDPHIGLLHRGTEKLIEAKTYLQAVPYFDRLDYCAPMNQEHAFCLAVERLAGIEVPKRGQLIRVLYSEIGRLLSHLLNVTTFAMDVGALTPPLWGFEAREKLMMFYERASGSRMHAAYFRPGGVHQDLPQKLVEDIGEFCRTFPSLLDDLDALVTGNRIFKQRTVDIGVVTLEDALAWGFSGVMVRGSGAPWDLRRAQPYECYSELDFDIPIGKHGDCYDRYVVRMEEMRQSNKIMLQCVDRLLKEAGPVASTDRKVSPPKRGEMKRSMEALIHHFKLYTEGFHVPEGEVYAAVEAPKGEFGVYLVGDGTNKPYRCKIRAPGFAHLQSMDFLCRGHMLADVSAVLGSLDIVFGEVDR

SEQ ID NO: 40 - protein, 192 aa, A. caulinodans
MTPSATKPLVSEAPKGIIDPSTGRPILSDDPFYKEINAELADKGFLVTTVDDLITWARTGSLMWMTFGLACCAVEMMQVSMPRYDVERFGFAPRASPRQSDVMIVAGTLTNKMAPALRKVYDQMPEPRYVISMGSCANGGGYYHYSYAVVRGCDRVVPVDIYVPGCPPTAEALLYGVLLLQRKIRRTGTIER